(19) United States
(12) Patent Application Publication
(10) Pub. No.: US 2025/0019456 A1
(43) Pub. Date: Jan. 16, 2025

(54) TARGETING IMMUNE INFILTRATION TO THE CENTRAL NERVOUS SYSTEM (CNS)

(71) Applicant: Ramot at Tel-Aviv University Ltd., Tel-Aviv (IL)

(72) Inventors: Lior MAYO, Tel-Aviv (IL); Adi TESSLER, Tel-Aviv (IL)

(73) Assignee: Ramot at Tel-Aviv University Ltd., Tel-Aviv (IL)

(21) Appl. No.: 18/900,759

(22) Filed: Sep. 29, 2024

Related U.S. Application Data
(63) Continuation of application No. PCT/IL2023/050338, filed on Mar. 30, 2023.
(60) Provisional application No. 63/325,662, filed on Mar. 31, 2022.

(51) Int. Cl. C07K 16/28; A61K 39/00; A61P 25/00; G01N 33/569

(52) U.S. Cl. CPC C07K 16/2896; A61P 25/00; G01N 33/56966; A61K 2039/505; C07K 2317/732; C07K 2317/734; G01N 2333/70596

(57) ABSTRACT

A method of treating an inflammatory disease or disorder of the central nervous system (CNS) in a subject in need thereof is disclosed. The method comprising administering to the subject a therapeutically effective amount of an agent capable of binding CD157 on CD157-expressing cells of the CNS, the agent capable of mediating a therapeutic effect.

### RELATED APPLICATIONS

This application is a Continuation of PCT Patent Application No. PCT/IL2023/050338 having International filing date of Mar. 30, 2023 which claims the benefit of priority under 35 USC § 119(e) of U.S. Provisional Patent Application No. 63/325,662 filed on Mar. 31, 2022.

PCT Patent Application No. PCT/IL2023/050338 is also related to co-filed PCT Patent Application No. PCT/IL2023/050337 entitled “TREATMENT OF BRAIN TUMORS BY TARGETING THE CHOLESTEROL PATHWAY IN ASTROCYES” (Attorney Docket No. 95283).

The contents of the above applications are all incorporated by reference as if fully set forth herein in their entirety.

### SEQUENCE LISTING STATEMENT

The XML file, entitled 100907SequenceListing.xml, created on Sep. 21, 2024, comprising 74,036 bytes, submitted concurrently with the filing of this application is incorporated herein by reference.

### FIELD AND BACKGROUND OF THE INVENTION

The present invention, in some embodiments thereof, relates to methods of treating and diagnosing diseases and conditions of the central nervous system (CNS) by determining the expression level of CD157 on CNS infiltrating cells.

Multiple sclerosis (MS) is an autoimmune disease of the CNS, affecting approximately 2.5 million people worldwide. The most common animal model for MS is experimental autoimmune encephalomyelitis (EAE), a murine model of MS. Both MS and EAE are characterized by infiltration of peripheral immune cells to the CNS, CNS inflammation and demyelination.

Infiltrating monocytes play an important role in disease progression, predominant the lesion area and their numbers are in correlation with disease severity. Moreover, studies have found that mice lacking the chemokine receptor CCR2, a key player in monocyte chemotactic migration, are resistant to EAE [Izikson L. et al., J Exp Med (2000) 192: 1075-1080].

One of the main challenges in the study of neuroinflammation is identifying a unique marker to distinguish infiltrating monocytes from resident microglial cells. Current separations of these two cell populations are based on morphology and on relative marker expression, e.g. by flow cytometry, such as of the common leukocyte antigen CD45^{hi/lo} [Ford A. L. et al., J Immunol (1995) 154(9): 4309-4321], of transmembrane protein 119 (Tmem119) [Bennett M. L. et al., Proc Natl Acad Sci USA (2016) 113: E1738-1746], or of CCR2 [Shi C. et. al., Nat Rev Immunol (2011) 11(11): 762-74]. Additional separations used to distinguish these two cell populations are based on generating bone marrow (BM) chimeras [Ajami B et al., Nat Neurosci (2007) 10(12):1538-1543] and on genetic modifications, such as microglial expression of the fractalkine receptor, Cx3cr1 [Jung S. et al., Mol Cell Biol (2000) 20: 4106-4114; Yona S, et al., Immunity (2013) 38(1):79-91]. However, each of these methods have their drawbacks, for example, cell morphology and CD45 expression may change with disease or injury, CCR2 expression may be unstable by downregulation once monocytes arrive to the injured site, and Cx3cr1 is also highly expressed by circulating monocytes (Ly6C^{lo}) and other tissue resident macrophages [Jung S. et al., Mol Cell Biol (2000), supra].

Monoclonal antibody (mAb) treatment has shown to be potentially effective in the treatment of different inflammatory diseases. Available mAb treatment for MS patients targets the adaptive immune system (i.e., T cells and B cells) [Wingerchuk D. M. et al., Mayo Clin Proc (2014) 89: 225-240]. Recent studies found that targeting CCR2⁺ monocytes depletes CCR2⁺Ly6c^{high} monocytes and improves clinical symptoms in EAE and tauopathy in mice [Mildner A. et al., Brain (2009) 132: 2487-2500; Ben-Yehuda H. et al., Mol Neurodegener (2021) 16: 39]. However, several therapies aimed to target CCR2 in MS failed during clinical trials due to lack of efficacy [Bose S. and Cho J., Arch Pharm Res (2013) 36: 1039-1050; Horuk R., Promiscuous drugs as therapeutics for chemokine receptors. Expert Rev Mol Med (2009) 11: e1].

CD157, also referred to as bone marrow stromal cell antigen-1 (BST-1), is a glycosyphosphatidylinositol (GPI)-linked glycoprotein encoded by a member of the NADase/ADP-ribosyl cyclase (cADPR) gene family. CD157 is expressed on the surface of cells in human and murine peripheral tissues, prevalently by myeloid cells. CD157 is not expressed in the healthy murine or human CNS and the role of CD157⁺ cells during neuroinflammation is unknown [Malavasi F. et al., Physiol Rev (2008) 88: 841-886; Quarona V. et al., Cytometry B Clin Cytom (2013) 84: 207-217; Ortolan et. al., Cell Biochem Funct (2002) 20: 309-322; Kaisho T. et al., Proc Natl Acad Sci USA (1994) 91: 5325-5329].

Additional background art includes Salmina et al., Messenger (2014) 3(1-2): 78-85 and Lopatina et al., Frontiers in Immunology (2020) 11:585294.

### SUMMARY OF THE INVENTION

According to an aspect of some embodiments of the present invention there is provided a method of treating an inflammatory disease or disorder of the central nervous system (CNS) in a subject in need thereof, the method comprising administering to the subject a therapeutically effective amount of an agent capable of binding CD157 on CD157-expressing cells of the CNS, the agent capable of mediating a therapeutic effect, thereby treating the inflammatory disease or disorder of the CNS in the subject.

According to an aspect of some embodiments of the present invention there is provided a method of treating an inflammatory disease or disorder of the central nervous system (CNS) in a subject in need thereof, the method comprising administering to or expressing in a CD157-expressing cell of the CNS of the subject an exogenous polynucleotide encoding an expression product capable of alleviating at least one symptom of the inflammatory disease or disorder, wherein the exogenous polynucleotide is under the transcriptional control of a cis acting regulatory element active specifically in a CD157-expressing cell, thereby treating the inflammatory disease or disorder of the CNS in the subject.

According to an aspect of some embodiments of the present invention there is provided a therapeutically effective amount of an agent capable of binding CD157 on CD157-expressing cells of the central nervous system (CNS) of a subject in need thereof for use in treating an inflammatory disease or disorder of the CNS, the agent capable of mediating a therapeutic effect.

According to an aspect of some embodiments of the present invention there is provided an exogenous polynucleotide encoding an expression product capable of alleviating at least one symptom of an inflammatory disease or disorder, wherein the exogenous polynucleotide is under the transcriptional control of a cis acting regulatory element specifically active in a CD157-expressing cell, for use in treating an inflammatory disease or disorder of the central nervous system (CNS) in a subject in need thereof.

According to an aspect of some embodiments of the present invention there is provided a method of diagnosing an inflammatory disease or disorder of the central nervous system (CNS) in a subject in need thereof, the method comprising analyzing a level of CD157-expressing cells in the CNS of the subject, wherein a level of the CD157-expressing cells above a predetermined threshold is indicative of the inflammatory disease or disorder of the CNS.

According to an aspect of some embodiments of the present invention there is provided a method of diagnosing and treating an inflammatory disease or disorder of the central nervous system (CNS) in a subject in need thereof, the method comprising: (a) analyzing a level of CD157-expressing cells in the CNS of the subject, wherein a level of the CD157-expressing cells above a predetermined threshold is indicative of the inflammatory disease or disorder of the CNS, and (b) administering to the subject a therapeutically effective amount of an agent capable of treating the inflammatory disease or disorder of the CNS in the subject, thereby diagnosing and treating the neuroinflammatory disease or disorder in the subject.

According to an aspect of some embodiments of the present invention there is provided a method of treating a subject having a tumor mass in the central nervous system (CNS), the method comprising: (a) analyzing a level of CD157-expressing cells in the CNS of the subject, wherein a level of the CD157-expressing cells above a predetermined threshold is indicative of presence of the tumor mass in the CNS, and (b) resecting the tumor mass, wherein the resecting is guided by the CD157-expressing cells, thereby treating the subject.

According to an aspect of some embodiments of the present invention there is provided a method of monitoring efficacy of a therapy to an inflammatory disease or disorder of the central nervous system (CNS) in a subject in need thereof, the method comprising analyzing a level of CD157-expressing cells in the CNS of the subject having been treated with an agent capable of treating the inflammatory disease or disorder of the CNS, and wherein a decrease in the level of CD157-expressing cells in the CNS of the subject from a predetermined threshold following the therapy is indicative of efficacy of the therapy, thereby monitoring efficacy of the therapy.

According to an aspect of some embodiments of the present invention there is provided an article of manufacture comprising an anti-CD157 antibody, and an additional agent for the treatment of an inflammatory disease or disorder of the CNS, being packaged in a packaging material and identified in print, in or on the packaging material for use in the treatment of the inflammatory disease or disorder of the CNS.

According to an aspect of some embodiments of the present invention there is provided a chimeric polynucleotide comprising a nucleic acid sequence encoding an expression product capable of alleviating at least one symptom of an inflammatory disease or disorder and another nucleic acid sequence comprising a cis acting regulatory element specifically active in a CD157-expressing cell.

According to an aspect of some embodiments of the present invention there is provided a composition of matter comprising the chimeric polynucleotide of some embodiments of the invention, and a particle encapsulating or attached to the chimeric polynucleotide.

According to an aspect of some embodiments of the invention there is provided a composition of matter comprising the chimeric polynucleotide of some embodiments of the invention, and a particle encapsulating the chimeric polynucleotide.

According to an aspect of some embodiments of the invention there is provided a composition of matter comprising the chimeric polynucleotide of some embodiments of the invention, and a particle attached to the chimeric polynucleotide.

According to some embodiments of the invention, analyzing is effected using an agent capable of binding CD157 on CD157-expressing cells in the CNS of the subject.

According to some embodiments of the invention, the agent capable of treating the inflammatory disease or disorder of the CNS comprises an agent capable of binding CD157 on the CD157-expressing cells of the CNS, the agent capable of mediating a therapeutic effect.

According to some embodiments of the invention, the agent capable of treating the inflammatory disease or disorder of the CNS is selected from the group consisting of an anti-inflammatory drug, an immunosuppressant drug, an immunomodulatory drug, a neuroprotective drug, a cognitive enhancing drug, a cognitive enhancing drug, a remyelination agent and an antitumor agent.

According to some embodiments of the invention, the antitumor agent is a localized cancer targeting therapy.

According to some embodiments of the invention, the agent is an antibody or fragment thereof.

According to some embodiments of the invention, the antibody comprises an anti-CD157 monoclonal antibody or fragment thereof.

According to some embodiments of the invention, the antibody or fragment thereof is conjugated to a therapeutic moiety.

According to some embodiments of the invention, the therapeutic moiety is an anti-inflammatory cytokine.

According to some embodiments of the invention, the therapeutic moiety is an agent capable of downregulating an expression of a protein upregulated in the neuroinflammatory disease or disorder.

According to some embodiments of the invention, the therapeutic moiety is a chemotherapeutic agent.

According to some embodiments of the invention, the antibody or fragment thereof is conjugated to a detection moiety.

According to some embodiments of the invention, the agent is a small molecule.

According to some embodiments of the invention, the cis acting regulatory element is a promoter.

According to some embodiments of the invention, the promoter comprises a bst1 promoter.

According to some embodiments of the invention, the exogenous polynucleotide is encapsulated in a particle.

According to some embodiments of the invention, the particle is a viral particle.

According to some embodiments of the invention, the particle is an Adeno-associated virus (AAV), Lentivirus (LV), adenovirus (Ad), retrovirus, or herpes simplex virus (HSV) particle.

According to some embodiments of the invention, the particle is an Adeno-associated virus (AAV) particle.

According to some embodiments of the invention, the expression product comprises a protein.

According to some embodiments of the invention, the protein is selected from the group consisting of an anti-inflammatory protein, a Neuronal survival factor, and a factor supporting remyelination.

According to some embodiments of the invention, the inflammatory disease or disorder of the CNS is a cancerous disease.

According to some embodiments of the invention, the cancerous disease is selected from the group consisting of Acoustic neuroma, Astrocytoma, Choroid plexus carcinoma, Craniopharyngioma, Embryonal tumor, Ependymoma, Glioblastoma, Glioma, Medulloblastoma, Meningioma, Oligodendroglioma, Pediatric brain tumor, Pineoblastoma, Pituitary tumor and Brain metastasis.

According to some embodiments of the invention, the inflammatory disease or disorder of the CNS is selected from the group consisting of Multiple Sclerosis (MS), Acute disseminated encephalomyelitis (ADEM), Acute Optic Neuritis (AON), Transverse Myelitis, Tauopathy, Neuromyelitis Optica (NMO), Autoimmune encephalitis, Viral encephalitis, Rasmussen's syndrome, Acute necrotizing encephalopathy of childhood (ANEC), Opsoclonus-myoclonus ataxia syndrome (OMAS), Parkinson's disease (PD), Alzheimer's disease (AD), Niemann's disease, Huntington's disease, Creutzfeldt-Jakob disease, Traumatic brain injury, Stroke, Spinal cord injury, neuroblastoma, Amyotrophic lateral sclerosis (ALS) and Spinal Muscular Atrophy (SMA).

According to some embodiments of the invention, the inflammatory disease or disorder of the CNS is Multiple Sclerosis (MS).

According to some embodiments of the invention, the Multiple Sclerosis (MS) is relapsing remitting multiple sclerosis (RRMS).

According to some embodiments of the invention, the Multiple Sclerosis (MS) is primary progressive multiple sclerosis (PPMS).

According to some embodiments of the invention, the Multiple Sclerosis (MS) is secondary progressive multiple sclerosis (SPMS).

According to some embodiments of the invention, the Multiple Sclerosis (MS) is an advanced or progressive multiple sclerosis.

According to some embodiments of the invention, the Multiple Sclerosis (MS) is an acute multiple sclerosis.

According to some embodiments of the invention, the CD157-expressing cells comprise monocytes.

According to some embodiments of the invention, the monocytes comprise CNS-infiltrating monocytes.

According to some embodiments of the invention, the CNS-infiltrating monocyte is positive for a CD157 marker (CD157+ cell).

According to some embodiments of the invention, the monocytes express at least one of CD11b, CD45, CCR2 and Ly6c.

According to some embodiments of the invention, the monocytes have a CD157⁺CD45⁺CCR2⁺, CD157⁺CD14⁺CD16⁺, CD157⁺CD14⁺CD16⁻, CD157⁺CX3CR1⁺ or a CD157⁺CD45⁺CD11b⁺ signature.

According to some embodiments of the invention, the additional agent for the treatment of an inflammatory disease or disorder of the CNS is selected from the group consisting of an anti-inflammatory drug, an immunosuppressant drug, an immunomodulatory drug, a neuroprotective drug and a cognitive enhancing drug.

According to some embodiments of the invention, the additional agent for the treatment of an inflammatory disease or disorder of the CNS is an anti-MS therapeutic agent.

According to some embodiments of the invention, the anti-MS therapeutic agent is selected from the group consisting of a beta interferon, glatiramer (Copaxone®), fingolimod (Gilenya®), natalizumab (Tysabri®), mitoxantrone (Novantrone®), teriflunimide (Aubagio®), BG-12 (Tecfidera®), alemtuzumab (Lemtrada®), daclizumab (Zinbryta®), ocrelizumab (Ocrevus®), amantadine (Symmetrel®), amitriptyline (Elavil®), nortriptyline, modafinil (Provigil®), and dalfampridine (Ampyra®).

According to some embodiments of the invention, the anti-CD157 antibody and the additional agent for the treatment of an inflammatory disease or disorder of the CNS are comprised in a single formulation.

According to some embodiments of the invention, the anti-CD157 antibody and the additional agent for the treatment of an inflammatory disease or disorder of the CNS are comprised in separate formulations.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G illustrate that CD157 is expressed in the CNS only during neuroinflammation. (FIGS. 1A-E) CD157 expression in naïve and EAE tissue. (FIG. 1A) quantitative PCR analysis of Bst1 expression in the brain and spinal cord (n=3). (FIG. 1B) CD157 expression in the brain and spinal cord quantified by immunoblot (n=2). (FIG. 1C) quantitative PCR analysis of Bst1 expression in the blood (n=2). (FIG. 1D) flow cytometry analysis of CD157 expression in the blood. (FIG. 1E) Histopathology analysis of lumbar spinal cord serial sections from EAE mouse, sister slides stained with Luxol Fast Blue and anti-CD157. Scale bar 200 μm. (FIGS. 1F-G) BST1 expression in human tissue. (FIG. 1F) BST1 expression in brain, spinal cord, and blood samples of healthy patients. (FIG. 1G) BST1 expression in brain samples of Multiple sclerosis patients or healthy controls. Statistical analysis by Student's t-test or one-way ANOVA. *P<0.05, **P<0.01, ***P<0.001, n.s.=not significant.

FIGS. 2A-E illustrate that CD157 is expressed in the CNS by CD11b⁺CD45^{high} monocytes. (FIG. 2A) immunofluorescence analysis of IBA1 (purple) and CD157 (yellow) in spinal cords isolated from EAE mice. Scale bar 40 mm. (FIG. 2B) FACS analysis of cells isolated from the CNS of naïve (left) and EAE (right) mice, presented as a heatmap of CD157 expression. (FIG. 2C) CD157 expression presented from CD11b⁺CD45^{low} (microglia), CD11b⁻CD45⁺ (lymphocytes) and CD11b⁺CD45^{high} (monocytes) cells, compared to isotype control. (FIGS. 2D-E) CD157 and CCR2 or Ly6C expression, gated from CD11b⁺CD45^{low} (left) or CD11b⁺CD45^{high} (right) cells (n=3). [symbol] Statistical analysis by Student's t-test or one-way ANOVA. *P<0.05, **P<0.01, ***P<0.001, n.s.=not significant.

FIGS. 3A-I illustrate that CD157 expression is a marker for infiltrating monocytes. (FIG. 3A) Fate mapping model schematic illustration. (FIG. 3B) blood analysis of tdTomato expression after TMX treatment and following 7 weeks. Presented as percentage of CD45⁺ cells. (FIG. 3C) Flow cytometry analysis of cells isolated from the CNS of naïve and EAE mice. (FIG. 3D) CD157 expression gated from eYFP⁺tdTomato⁺ and eYFP⁺tdTomato⁻ cells, presented as histogram and percentage of cells (n=3). (FIG. 3E) Schematic illustration of bone marrow transplantation model. (FIG. 3F) eYFP expression in the blood of bone marrow-transplanted mice, as assessed by flow cytometry. (FIG. 3G) Flow cytometry assessment of CD11b⁺eYFP⁺ cells to the CNS of naïve and EAE transplanted mice. (FIG. 3H) CD157 expression gated from CD11b⁺eYFP⁺ cells, presented as histogram and percentage of cells (n=3). (FIG. 3I) Co-localization of eYFP and CD157 presented by immunofluorescence staining. Scale bar 20 mm. Statistical analysis by Student's t-test or one-way ANOVA. *P<0.05, **P<0.01, ***P<0.001, n.s.=not significant.

FIGS. 4A-I illustrate that targeting CD157⁺ monocytes ameliorates EAE development. (FIG. 4A) complement-dependent cytotoxicity (CDC) and (FIG. 4B) antibody-dependent cellular cytotoxicity (ADCC) mediated by anti-CD157 mAbs in-vitro. Tested on RAW264.7 murine macrophages. Presented as percentage of cell death (n=3). (FIG. 4C) Administration of 100 μg anti-CD157 antibody in-vivo significantly depletes CCR2⁺Ly6C⁺ circulating monocytes. FIGS. 4D-G: EAE clinical scores (FIG. 4D), linear regression (FIG. 4E), weight loss (FIG. 4F) and percentage of healthy mice (FIG. 4G) in EAE mice treated with anti-CD157 mAbs or isotype control from day 7 post-immunization. Representative data from one out of three independent experiments (n=6). (FIG. 4H) Histopathological analysis of spinal cords isolated from EAE mice treated with anti-CD157 mAbs or isotype control. Demyelination assessed using Luxol Fast Blue staining (FIG. 4I) Recruitment of inflammatory monocytes (CD11b⁺CD45^{high}) from EAE mice treated with anti-CD157 mAbs or isotype control, assessed by flow cytometry (left) and presented as percentage of cells (right). Representative data from one out of two independent experiments (n=6). Statistical analysis by Student's t-test, one-way ANOVA, or two-way ANOVA. *P<0.05, **P<0.01, ***P<0.001, n.s.=not significant.

FIGS. 5A-G illustrate that CD157⁺ monocytes promote disease progression in the progressive phase of EAE. (FIG. 5A) clinical score of NOD-C57B16 F1 hybrid EAE mice. (FIG. 5B) Flow analysis of CD11b⁺CD157⁻ and CD11b⁺CD157⁺ cells isolated from the CNS of naïve and EAE NOD-C57B16 F1 mice (FIG. 5C) CD157 expression in the CNS of naïve and EAE NOD-C57B16 F1 mice, assessed by flow cytometry, presented as heatmap for CD157 expression. (FIGS. 5D-E) Administration of 50 μg anti-CD157 mAbs, every other day, ameliorates disease progression in NOD-C57B16 F1 EAE. representative data from one out of two independent experiments (n=4). (FIG. 5F) Histopathological analysis of spinal cords isolated from EAE NOD-C57B16 F1 mice treated with anti-CD157 mAbs or isotype control. Demyelination was assessed using Luxol Fast Blue staining (top) and cell infiltration by H&E (bottom) (FIG. 5G) Recruitment of inflammatory monocytes (CD11b⁺CD45^{high}) from EAE NOD-C57B16 F1 mice treated with anti-CD157 mAbs or isotype control, assessed by flow cytometry (left) and presented as percentage of cells (right). Representative data from one out of two independent experiments (n=6). Statistical analysis by Student's t-test, one-way ANOVA, or two-way ANOVA. *P<0.05, **P<0.01, ***P<0.001, n.s.=not significant.

### DESCRIPTION OF SPECIFIC EMBODIMENTS OF THE INVENTION

The present invention, in some embodiments thereof, relates to methods of treating and diagnosing diseases and conditions of the central nervous system (CNS) by determining the expression level of CD157 on CNS infiltrating cells.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Monocytes play an important role in neuroinflammation and other CNS related diseases. The cell surface protein CD157 is expressed in human and murine peripheral tissue, prevalently by myeloid cells. CD157 is not expressed in the healthy murine or human CNS.

While reducing the present invention to practice, the present inventors have uncovered that CD157 is a novel marker which can be used to identify infiltrating monocytes to the CNS and is not expressed by resident microglial cells (see Example 1 of the Examples section which follows). Specifically, CD157-expressing monocytes in the CNS are CD11b⁺CD45⁺⁺ cells, these cells also typically express CCR2 and/or Ly6C (see Example 2 of the Examples section which follows). Targeting CD157⁺ cells during multiple sclerosis (MS) in an animal model (EAE murine model) significantly depleted circulating monocytes and was found to be neuroprotective in acute and progressive models of EAE (see Example 4 of the Examples section which follows). Furthermore, CD157 was found to be a marker for infiltrating monocytes in progressive MS (see Example 5 of the Examples section which follows). Taken together, CD157 was established as a novel marker which can be used to identify inflammatory monocytes in the CNS, and which can be used as a therapeutic target for patients with MS and other neuroinflammatory diseases and disorders.

According to an aspect of some embodiments of the invention there is provided a method of treating an inflammatory disease or disorder of the central nervous system (CNS) in a subject in need thereof, the method comprising administering to the subject a therapeutically effective amount of an agent capable of binding CD157 on CD157-expressing cells of the CNS, the agent capable of mediating a therapeutic effect, thereby treating the inflammatory disease or disorder of the CNS in the subject.

According to an aspect of some embodiments of the invention there is provided a therapeutically effective amount of an agent capable of binding CD157 on CD157-expressing cells of the central nervous system (CNS) of a subject in need thereof for use in treating an inflammatory disease or disorder of the CNS, the agent capable of mediating a therapeutic effect.

As used herein, the term “treating” includes abrogating, substantially inhibiting, slowing or reversing the progression of a pathology (e.g., an inflammatory disease or disorder of the central nervous system (CNS)), causing the reduction, remission, or regression of a pathology, substantially ameliorating clinical or aesthetical symptoms of a pathology or substantially preventing the appearance of clinical or aesthetical symptoms of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

As used herein the phrase “an inflammatory disease or disorder of the central nervous system (CNS)” refers to an inflammatory response within the brain or spinal cord. Typically such an inflammation is mediated by the production of cytokines, chemokines, reactive oxygen species, and secondary messengers produced by resident CNS glia, and peripherally derived immune cells, specifically monocytes.

According to one embodiment, the inflammatory disease or disorder of the CNS is associated with local accumulation of fluids, plasma proteins, and white blood cells.

According to one embodiment, the inflammatory disease or disorder of the CNS may be initiated by physical injury, trauma, infection, stress or a local immune response (e.g., in the CNS).

Inflammation is an aspect of many diseases and disorders of the CNS, including but not limited to, physical injuries or traumas, diseases related to immune disorders, pathogens (e.g. viral and bacterial infections), damaged cells, or irritants, and includes secretion of cytokines and more particularly of pro-inflammatory cytokines, i.e. cytokines which are produced predominantly by activated immune cells (e.g. microglia) but also by other cells in the CNS (e.g. astrocytes, endothelial cells). Exemplary pro-inflammatory cytokines include, but are not limited to, IL-Iβ, IL-6, CXCL1, CXCL2 and TNF-α. Such pro-inflammatory cytokines are generally involved in the amplification of the inflammatory reaction, such as in activation of endothelial cells, platelet deposition, and tissue edema (e.g. in acute inflammation), or in sustained activation of microglia cells and recruitment of other immune cells into the brain (e.g. in chronic inflammation).

According to one embodiment, the inflammation can be a sterile inflammation (i.e., as a result of an injury, trauma or stroke) or a pathogenic inflammation (i.e., caused by a pathogen such as a bacteria, virus or fungus).

According to one embodiment, the inflammatory disease or disorder of the CNS is selected from the group consisting of Multiple Sclerosis (MS), Acute disseminated encephalomyelitis (ADEM), Acute Optic Neuritis (AON), Transverse Myelitis, Neuromyelitis Optica (NMO), Autoimmune encephalitis, Viral encephalitis, Rasmussen's syndrome, Acute necrotizing encephalopathy of childhood (ANEC), Opsoclonus-myoclonus ataxia syndrome (OMAS), Parkinson's disease (PD), Alzheimer's disease (AD), Niemann's disease, Huntington's disease, Creutzfeldt-Jakob disease, Traumatic brain injury, Stroke, Spinal cord injury, neuroblastoma, Amyotrophic lateral sclerosis (ALS) and Spinal Muscular Atrophy (SMA).

According to some embodiments of the invention, the inflammatory disease or disorder of the CNS is Multiple Sclerosis (MS).

As used herein the phrase “multiple sclerosis” (“MS”) refers to a pathology characterized by presence of at least two neurological attacks affecting the central nervous system (CNS) and accompanied by demyelinating lesions on brain magnetic resonance imaging (MRI).

The pathobiology of MS includes inflammatory and neurodegenerative mechanisms that affect both white and gray matter. The main pathologic findings in MS are the presence of infiltrating mononuclear cells, predominantly T lymphocytes and macrophages, that surpass the blood brain barrier and induce an active inflammation within the brain and spinal cord, attacking the myelin and resulting in gliotic scars and axonal loss. These inflammatory (acute and chronic) processes can be visualized by brain and spinal cord magnetic resonance imaging (MRI) as hyperintense T2 or hypointense T1 lesions. These mechanisms underlie the relapsing-remitting and often eventually progressive, courses of MS.

In about 85% of the patients with diagnosis of MS, the disease course is relapsing-remitting definite MS (RRMS), which is characterized by attacks during which new neurological symptoms and signs appear, or existing neurological symptoms and signs worsen. In about 15% of the patients the disease has a primary progressive course, characterized by gradual onset of neurological symptoms that progress over time. In a subset of patients (about 40%), the disease has a secondary progressive course, i.e., it is first characterized by relapses and remission and then gradually progresses.

Various immunomodulatory drugs have been shown to reduce the number and severity of acute attacks, and thereby to decrease the accumulation of neurological disability. Currently, there are several approved MS disease-modifying treatments (DMTs) with varying degrees of efficacy for reducing relapse risk and preserving neurological function. Available DMTs differ with respect to the route and frequency of administration, tolerability and likelihood of treatment adherence, common adverse effects, risk of major toxicity, and pregnancy-related risks. This of utmost importance to understand the benefit-risk profiles of these therapies to establish logical and safe treatment plans for MS patients.

According to some embodiments of the invention, the Multiple Sclerosis (MS) is an advanced or progressive multiple sclerosis.

According to some embodiments of the invention, the Multiple Sclerosis (MS) is an acute multiple sclerosis.

According to some embodiments of the invention, the inflammatory disease or disorder of the CNS is a cancerous disease.

According to some embodiments of the invention, the cancerous disease is selected from the group consisting of Acoustic neuroma, Astrocytoma, Choroid plexus carcinoma, Craniopharyngioma, Embryonal tumor, Ependymoma, Glioblastoma, Glioma, Medulloblastoma, Meningioma, Oligodendroglioma, Pediatric brain tumor, Pineoblastoma, Pituitary tumor and Brain metastasis.

As used herein the term “CD157”, also referred to as Bone Marrow Stromal Cell Antigen 1 (BST1), refers to the gene encoding the CD157 molecule having the gene symbol “BST1”. An exemplary CD157 transcript is provided in GenBank Accession No. NM_004334.3 (SEQ ID NO: 19). An exemplary CD157 protein is provided in GenBank Accession No. NP_004325.2 (SEQ ID NO: 20).

The phrase “CD157-expressing cells” refers to cells having CD157 protein on the cell surface which can be detected by cell surface protein detection assays, such as but not limited to, binding assays, flow cytometry (e.g. FACS), Enzyme-Linked Immunosorbent Assay (ELISA), and Western Blot. Typically CD157 is anchored to the cell surface as a glycosylphosphatidylinositol-anchored molecule.

According to some embodiments of the invention, CD157-expressing cells comprise myeloid cells.

According to some embodiments of the invention, CD157-expressing cells comprise monocytes.

According to some embodiments of the invention, CD157-expressing cells comprise granulocytes.

According to some embodiments of the invention, CD157-expressing cells comprise CNS-infiltrating monocytes.

According to some embodiments of the invention, CD157-expressing cells comprise CNS-infiltrating granulocytes.

As used herein the phrase “CNS-infiltrating monocytes” refers to monocytes which are capable of crossing the blood brain barrier (BBB) and are present in the tissue affected by the inflammatory disease or disorder of the CNS.

As used herein the phrase “CNS-infiltrating granulocytes” refers to granulocytes which are capable of crossing the blood brain barrier (BBB) and are present in the tissue affected by the inflammatory disease or disorder of the CNS.

According to some embodiments of the invention, the CNS-infiltrating monocytes express CD157 and in addition at least one of the CD11b, CD45, CX3CR1, CCR2 and/or CD14 markers.

According to some embodiments of the invention, the CNS-infiltrating monocytes express at least one of the CD11b, CD45, CCR2 and Ly6c markers.

As used herein the term “CD11b” refers to the product (mRNA or protein) of the integrin subunit alpha M gene (gene symbol: ITGAM).

As used herein the term “CD45” refers to the product (mRNA or protein) of the protein tyrosine phosphatase receptor type C gene (gene symbol: PTPRC).

As used herein the term “CCR2” refers to the product (mRNA or protein) of the C-C motif chemokine receptor 2 gene (gene symbol: CCR2).

As used herein the term “CX3CR1” refers to the product (mRNA or protein) of the C-C motif chemokine receptor 2 gene (gene symbol: Cx3cr1).

As used herein the term “Ly6c” refers to the product (mRNA or protein) of the lymphocyte antigen 6 complex, locus C1 (mouse gene symbol: Ly6c1). The equivalent marker in human is the combination of CD14 and CD16 markers (e.g., CD14⁺CD16⁺ cells).

It should be noted that cells which express the above described marker(s) (e.g., CD11b, CD45, CX3CR1, CCR2, CD14 and/or CD16) are often referred to as being positive (+) for expression of these markers, and can be identified, e.g., as having the expression signature CD157+, CD11b⁺, CD45⁺, CCR2⁺, CD14⁺ and/or CD16+.

According to some embodiments of the invention, the CNS-infiltrating monocytes have a CD157⁺CD45⁺CCR2⁺Ly6c⁺ or CD157⁺CD45⁺CD11b⁺ signature.

According to some embodiments of the invention, the CNS-infiltrating monocytes have a CD157⁺CD14⁺CD16⁻ or CD157⁺CD14⁺CD16⁺ or a CD157⁺CD45⁺CD11b⁺ or CD157⁺CX3CR1⁺ or a CD157⁺CD45⁺CCR2⁺ signature.

The phrase “agent capable of binding CD157” refers to a molecule that is capable of binding to CD157 on the cell surface under physiological conditions.

As mentioned, the agent is capable of mediating a therapeutic effect. It should be noted that the agent can either directly mediate the therapeutic effect, i.e., treat the inflammatory disease or disorder of the CNS, or it can indirectly mediate the therapeutic effect by being a targeting moiety for a therapeutic moiety capable of treating the inflammatory disease or disorder of the CNS.

According to one embodiment, the agent capable of binding CD157 is an antibody or fragment thereof.

According to some embodiments of the invention, the antibody is capable of binding CD157 on CD157-expressing cells of the CNS.

The term “antibody” as used in this invention includes intact molecules as well as functional fragments and/or mimetics thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)2 fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Antibody mimetics are small proteins (usually less than 20 kDa) that mimic CDR display within antibody Fab fragments but lack the Fc. Antibody mimetics can be obtained using methods like phage display. To increase their in vivo stability antibody mimetics can be conjugated to specific sequences by chemical conjugation or genetic fusion (reviewed in Angeline N Ta & Brian R McNaughton. 2017. FUTURE MEDICINAL CHEMISTRY Vol. 9, NO. 12; “Antibody and antibody mimetic immunotherapeutics”; which is fully incorporated herein by reference in its entirety) Once antibodies are obtained, they may be tested for activity, for example via ELISA.

According to some embodiments of the invention, the antibody comprises an anti-CD157 monoclonal antibody or fragment thereof.

According to some embodiments of the invention, the agent capable of binding CD157 on the CD157-expressing cells comprises an aptamer.

As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

According to some embodiments of the invention, the agent capable of binding CD157 on the CD157-expressing cells (e.g., the antibody or fragment thereof) is conjugated to a therapeutic moiety (an immune-conjugate molecule, e.g., an antibody drug conjugate) capable of treating an inflammatory disease or disorder of the central nervous system (CNS).

According to some embodiments of the invention, the immune-conjugate molecule can be an isolated molecule such as a soluble and/or a synthetic molecule.

The therapeutic moiety can be an agent capable of treating an inflammatory disease or disorder of the central nervous system, for example, a cytotoxic moiety, a toxic moiety, a polypeptide, a cytokine moiety or a second antibody moiety comprising a different specificity to the antibodies of the invention.

Non-limiting examples of therapeutic moieties which can be conjugated to the antibody of the invention are provided in Table 1, hereinbelow.

TABLE 1

| | Amino acid sequence | Nucleic acid sequence |
|---|---|---|
| | (GenBank Accession | (GenBank Accession |
| Therapeutic moiety | No.)/SEQ ID NO: | No.)/SEQ ID NO: |
| Pseudomonas exotoxin | ABU63124/21 | EU090068/22 |
| Diphtheria toxin | AAV70486/23 | AY820132.1/24 |
| interleukin 2 | CAA00227/25 | A02159/26 |
| CD3 | P07766/27 | X03884/28 |
| CD16 | NP_000560.5/29 | NM_000569.6/30 |
| interleukin 4 | NP_000580.1/31 | NM_000589.2/32 |
| HLA-A2 | P01892/33 | K02883/34 |
| Ricin toxin | EEF27734/35 | EQ975183/36 |

Table 1.

The functional moiety (e.g., the therapeutic moiety of some embodiments of the invention) may be attached or conjugated to the antibody of the invention in various ways, depending on the context, application and purpose.

When the functional moiety is a polypeptide (e.g., a therapeutic polypeptide capable of mediating a therapeutic effect), the immune-conjugate may be produced by recombinant means. For example, the nucleic acid sequence encoding the therapeutic polypeptide may be ligated in-frame with the nucleic acid sequence encoding the antibody of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like.

According to some embodiments of the invention, the therapeutic moiety is connected to the agent (e.g., antibody) via a cleavable linker. Examples include, but are not limited to, an acid-labile linker, a protease cleavable linker, photoremovable linker, or a linker that is connected by a disulphide linkage. It should be noted that cleavable linkers can be released in an inflammatory site, or in the region of a tumor, or can be released in a more acidic environment.

According to some embodiments of the invention, the therapeutic moiety is connected to the agent (e.g., antibody) via a non-cleavable linker.

Exemplary methods for conjugating peptide moieties (e.g., a therapeutic or detectable moiety) to the antibody of some embodiments of the invention include, but are not limited to, SPDP conjugation (e.g., as described in Cumber et al. (1985, Methods of Enzymology 112: 207-224); Glutaraldehyde conjugation (e.g., as described in G. T. Hermanson (1996, “Antibody Modification and Conjugation, in Bioconjugate Techniques, Academic Press, San Diego); and Carbodiimide conjugation (e.g., as described in J. March, Advanced Organic Chemistry: Reaction's, Mechanism, and Structure, pp. 349-50 & 372-74 (3d ed.), 1985; B. Neises et al. (1978), Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)].

According to some embodiments of the invention, the therapeutic moiety is an anti-inflammatory cytokine.

Non-limiting examples of suitable an anti-inflammatory cytokines include, but are not limited to anti-inflammatory interleukins include interleukin (IL)-1 receptor antagonist, IL-4, IL-6, IL-13, IL-19, IL-27 and IL-35, and TGF-β (transforming growth factor beta).

According to some embodiments of the invention, the therapeutic moiety is an agent capable of downregulating an expression of a protein upregulated in the neuroinflammatory disease or disorder.

Non-limiting examples of proteins which are upregulated in the neuroinflammatory disease or disorder include, but are not limited to IL-1β (interleukin-1-beta), IL-12, IL-6, IL-23, TNF-α (Tumor Necrosis Factor alpha), colony stimulating factor 2 (CSF2; also known as GM-CSF), Interferons, nitric oxide synthase 2 (NOS2; also known as NOS; INOS; NOS2A; HEP-NOS).

According to some embodiments of the invention, the therapeutic moiety is a chemotherapeutic agent.

It should be noted that the choice of chemotherapeutic agents depends on the type of the neuroinflammatory disease or disorder of the CNS.

Following is a non-limiting list of chemotherapeutic agents which can be used along with the method of some embodiments of the invention: Aldesleukin; Alemtuzumab; alitretinoin; allopurinol; altretamine; amifostine; amifostine; anastrozole; anastrozole; anastrozole; arsenic trioxide; Asparaginase; BCG Live; bexarotene capsules; bexarotene gel; bleomycin; bleomycin; busulfan intravenous; busulfan oral; calusterone; capecitabine; capecitabine; carboplatin; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin, actinomycin D; Darbepoetin alfa; daunorubicin liposomal; daunorubicin; Denileukin diftitox; dexrazoxane; docetaxel; doxorubicin; doxorubicin liposomal; Dromostanolone Propionate; Elliott's B Solution; epirubicin; Epoetin alfa; estramustine; etoposide phosphate; etoposide, VP-16; exemestane; Filgrastim; floxuridine (intraarterial); fludarabine; fluorouracil, 5-FU; fulvestrant; gemcitabine; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine, CCNU; meclorethamine, nitrogen mustard; megestrol acetate; melphalan, L-PAM; melphalan, L-PAM; mercaptopurine, 6-MP; mesna; methotrexate; methoxsalen; mitomycin C; mitotane; mitoxantrone; nandrolone phenpropionate; Nofetumomab; Oprelvekin; oxaliplatin; paclitaxel; pamidronate; pegademase; Pegaspargase; Pegfilgrastim; pentostatin; pipobroman; plicamycin, mithramycin; porfimer sodium; procarbazine; quinacrine; Rasburicase; Rituximab; Sargramostim; Streptozocin; talc; tamoxifen; temozolomide; teniposide, VM-26; testolactone; thioguanine, 6-TG; thiotepa; topotecan; toremifene; Tositumomab; Trastuzumab; tretinoin, ATRA; Uracil Mustard; Valrubicin; vinblastine; vincristine; vinorelbine; and zoledronate.

According to some embodiments of the invention, the agent capable of treating the inflammatory disease or disorder of the CNS is selected from the group consisting of an anti-inflammatory drug, an immunosuppressant drug, an immunomodulatory drug, a neuroprotective drug, a cognitive enhancing drug, a remyelination agent and an antitumor agent.

Non-limiting examples of anti-inflammatory drugs which can be used along with the method of some embodiments of the invention include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Non-limiting examples of immunosuppressant drug which can be used along with the method of some embodiments of the invention include Corticosteroids, such as prednisone; Biologics such as adalimumab (Humira®) and infliximab (Remicade®); Calcineurin inhibitors such as tacrolimus (Envarsus XR® or Protopic) and cyclosporine (Gengraf®, Neoral® or Sandimmune®); Inosine monophosphate dehydrogenase (IMDH) inhibitors such as mycophenolate mofetil (CellCept®); Janus kinase inhibitors such as tofacitinib (Xeljanz®); Mechanistic target of rapamycin (mTOR) inhibitors such as sirolimus (Rapamune®); Monoclonal antibodies such as basiliximab (Simulect®); and FK506 (Tacrolimus).

Non-limiting examples of suitable immunomodulatory drugs which can be used along with the method of some embodiments of the invention, include Thalidomide (Thalomid), lenalidomide (Revlimid), and pomalidomide (Pomalyst).

Non-limiting examples of neuroprotective drugs which can be used along with the method of some embodiments of the invention include Glutamate blockers such as polyarginine R18 and NA-1 (TAT-NR2B9c); Glutamate blockers/NMDA channels blocker such as Magnesium sulfate (MgSO4); Statins such as Atorvastatin, Mevastatin, Rosuvastatin and Simvastatin; Hormones such as Melatonin; Hematopoietic growth factors such as Erythropoietin; Free Radical Scavengers such as NXY-059, PEG-SOD, Tempol, hydroxystilbene oxyresveratrol; Mucolytic agents such as NAC; Blockers of beta-adrenergic receptors such as Esmolol, propranolol, labetalol, metoprolol, atenolol or carvedilol; COX-2 inhibitors such as Flavocoxid, NS-398, Valdecoxib, Celecoxib; and Herbal medicine such as Curcumin (Yunes Panahi et al., J Pharmacopuncture. 2018 December; 21(4): 226-240).

Non-limiting examples of cognitive enhancing drugs which can be used along with the method of some embodiments of the invention include, donepezil (Aricept®), rivastigmine tartrate (Exelon®), galantamine HBr (Reminyl®), memantine (Namenda®); and modafinil (Provigil®).

Non-limiting examples of remyelination agents which can be used along with the method of some embodiments of the invention include, antagonists of M1/M3 muscarinic acetylcholine receptors with subsequent stimulation of oligodendrocyte differentiation such as Benztropine; Activators of eIF2, TX/RXR, and cholesterol signaling such as Miconazole and clobetasol; agents that decreases oxidative stress and promote oligodendrocyte maturation and myelin synthesis such as Olesoxime; agents that stimulate proliferation and maturation of oligodendrocytes, increase neurotrophic factors, and inhibit activated microglia, astrocytes, and T lymphocytes such as Quetiapine fumarate; and rHIgM22 which binds to the surface of oligodendrocytes and promote myelin repair (Danielle E. Harlow et al., Front Neurol. 2015; 6: 257).

The antitumor agent which can be used along with the method of some embodiments of the invention can be for example a chemotherapeutic agent, a radiation therapy agent, and/or an anti-angiogenesis agent (e.g., anti-VEGF antibody).

According to some embodiments of the invention, the antitumor agent is a localized cancer targeting therapy, such as radiotherapy via a specialized bead.

According to some embodiments of the invention, the agent is a small molecule.

According to some embodiments of the invention, the small molecule is an anti-MS therapeutic agent.

According to some embodiments of the invention, the anti-MS therapeutic agent is selected from the group consisting of a beta interferon, glatiramer (Copaxone®), fingolimod (Gilenya®), natalizumab (Tysabri®), mitoxantrone (Novantrone®), teriflunimide (Aubagio®), BG-12 (Tecfidera®), alemtuzumab (Lemtrada®), daclizumab (Zinbryta®), ocrelizumab (Ocrevus®), amantadine (Symmetrel®), amitriptyline (Elavil®), nortriptyline, modafinil (Provigil®), and dalfampridine (Ampyra®).

According to an aspect of some embodiments of the invention there is provided an article of manufacture comprising an agent capable of binding CD157 on CD157-expressing cells of the CNS, and an additional agent for the treatment of an inflammatory disease or disorder of the CNS, being packaged in a packaging material and identified in print, in or on the packaging material for use in the treatment of the inflammatory disease or disorder of the CNS.

According to an aspect of some embodiments of the invention there is provided an article of manufacture comprising an anti-CD157 antibody, and an additional agent for the treatment of an inflammatory disease or disorder of the CNS, being packaged in a packaging material and identified in print, in or on the packaging material for use in the treatment of the inflammatory disease or disorder of the CNS.

According to some embodiments of the invention, the additional agent for the treatment of an inflammatory disease or disorder of the CNS is selected from the group consisting of an anti-inflammatory drug, an immunosuppressant drug, an immunomodulatory drug, a neuroprotective drug, a cognitive enhancing drug, a remyelination agent and an antitumor agent.

According to some embodiments of the invention, the additional agent for the treatment of an inflammatory disease or disorder of the CNS is an anti-MS therapeutic agent.

According to some embodiments of the invention, the agent capable of binding CD157 on CD157-expressing cells of the CNS and the additional agent for the treatment of an inflammatory disease or disorder of the CNS are comprised in a single formulation.

According to some embodiments of the invention, the anti-CD157 antibody and the additional agent for the treatment of an inflammatory disease or disorder of the CNS are comprised in a single formulation.

According to some embodiments of the invention, the agent capable of binding CD157 on CD157-expressing cells of the CNS and the additional agent for the treatment of an inflammatory disease or disorder of the CNS are comprised in separate formulations.

According to some embodiments of the invention, the anti-CD157 antibody and the additional agent for the treatment of an inflammatory disease or disorder of the CNS are comprised in separate formulations.

According to an aspect of some embodiments of the invention there is provided a method of treating an inflammatory disease or disorder of the central nervous system (CNS) in a subject in need thereof, the method comprising administering to or expressing in a CD157-expressing cell of the CNS of the subject an exogenous polynucleotide encoding an expression product capable of alleviating at least one symptom of the inflammatory disease or disorder, wherein the exogenous polynucleotide is under the transcriptional control of a cis acting regulatory element active specifically in a CD157-expressing cell, thereby treating the inflammatory disease or disorder of the CNS in the subject.

According to an aspect of some embodiments of the invention there is provided an exogenous polynucleotide encoding an expression product capable of alleviating at least one symptom of an inflammatory disease or disorder, wherein the exogenous polynucleotide is under the transcriptional control of a cis acting regulatory element specifically active in a CD157-expressing cell, for use in treating an inflammatory disease or disorder of the central nervous system (CNS) in a subject in need thereof.

The term “exogenous” as used herein refers to a heterologous nucleic acid sequence which may not be naturally expressed within the subject (e.g., a nucleic acid sequence from a different organism, e.g., species) or which overexpression in the subject is desired.

According to some embodiments of the invention, the cis-acting regulatory element is a promoter which is specifically active in CD157-expressing cells such as the bst1 promoter.

According to some embodiments of the invention, the cis-acting regulatory element comprises a bst1 promoter.

According to some embodiments of the invention, the bst1 promoter is as set forth by SEQ ID NO: 37.

According to some embodiments of the invention, the bst1 promoter is as set forth by SEQ ID NO: 38.

According to some embodiments of the invention, the expression product is an mRNA or a protein capable of alleviating at least one symptom of the inflammatory disease or disorder.

According to some embodiments of the invention, the expression product comprises a protein.

According to some embodiments of the invention, the protein is selected from the group consisting of an anti-inflammatory protein, a neuronal survival factor, and a factor supporting remyelination.

Non-limiting examples of suitable anti-inflammatory proteins include anti-inflammatory interleukins such as interleukin (IL)-1 receptor antagonist, IL-4, IL-6, IL-13, IL-19, IL-27 and IL-35 and transforming growth factor beta (TGFβ).

Non-limiting examples of suitable neuronal survival factors include neuronal growth factor (NGF), neurotrophic factor (BDNF), neurotrophin-3 (NT-3), neurotrophin-4 (NT-4), TGFβ, Insulin-like growth factor-I (IGF-I), Ciliary neurotrophic factor (CNTF), Connective tissue growth factor (CTGF).

Non-limiting examples of suitable factors supporting remyelination include brain derived neurotrophic factor (BDNF), ciliary neurotrophic factor (CNTF), nerve growth factor (NGF), Activin A (INHBA), Galectin 3, Leukemia Inhibitory Factor (LIF), Biotin, Leucine-rich repeat and immunoglobulin domain-containing protein (Lingo-1) inhibiting/neutralizing moieties, and Fibroblast growth factor 2 (FGF2).

According to some embodiments of the invention, the exogenous polynucleotide is encapsulated in a particle.

According to some embodiments of the invention, the particle is an Adeno-associated virus (AAV), Lentivirus (LV), adenovirus (Ad), retrovirus, or herpes simplex virus (HSV) particles.

According to an aspect of some embodiments of the invention there is provided a chimeric polynucleotide comprising a nucleic acid sequence encoding an expression product capable of alleviating at least one symptom of an inflammatory disease or disorder and another nucleic acid sequence comprising a cis acting regulatory element specifically active in a CD157-expressing cell.

As used herein the term “chimeric polynucleotide” refers to a polynucleotide which comprises at least two distinct nucleic acid sequences, wherein the combination of same in the chimeric polynucleotide is not found in nature.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to some embodiments of the invention, the chimeric polynucleotide of some embodiments of the invention is an isolated polynucleotide.

The term “isolated” refers to at least partially separated from the natural environment e.g., from a human cell.

According to some embodiments of the invention, the cis acting regulatory element is heterologous to the nucleic acid sequence encoding the expression product capable of alleviating at least one symptom of an inflammatory disease or disorder.

According to some embodiments of the invention, the nucleic acid sequence encoding the expression product capable of alleviating at least one symptom of an inflammatory disease or disorder is under the transcriptional regulation of the cis-acting regulatory element specifically active in a CD157-expressing cell.

According to some embodiments of the invention, the cis acting regulatory element is a promoter.

According to some embodiments of the invention, the promoter comprises a bst1 promoter.

According to an aspect of some embodiments of the invention there is provided a composition of matter comprising the chimeric polynucleotide of some embodiments of the invention, and a particle encapsulating or attached to the chimeric polynucleotide.

According to some embodiments of the invention, the particle of the composition of matter is a viral particle.

Non-limiting examples of viral particles which can be used include Adeno-associated virus (AAV), Lentivirus (LV), adenovirus (Ad), retrovirus, or herpes simplex virus (HSV) particles.

According to an aspect of some embodiments of the invention there is provided a method of diagnosing an inflammatory disease or disorder of the central nervous system (CNS) in a subject in need thereof, the method comprising analyzing a level of CD157-expressing cells in the CNS of the subject, wherein a level of the CD157-expressing cells above a predetermined threshold is indicative of the inflammatory disease or disorder of the CNS.

As used herein the term “diagnosing” refers to determining presence or absence of a pathology (e.g., an inflammatory disease or disorder of the central nervous system), classifying a pathology or a symptom, determining a severity of the pathology, monitoring pathology progression, forecasting an outcome of a pathology and/or prospects of recovery and screening of a subject for a specific disease.

According to some embodiments of the invention, analyzing is effected using an agent capable of binding CD157 on CD157-expressing cells in the CNS of the subject.

According to one embodiment, the agent capable of binding CD157 on the CD157-expressing cells is an antibody or fragment thereof.

According to one embodiment, the agent capable of binding CD157 on the CD157-expressing cells is an antibody mimetic.

According to some embodiments of the invention, the agent capable of binding CD157 on the CD157-expressing cells comprises an aptamer.

According to the method of some embodiments of the invention, the level of CD157-expressing cells in the CNS is analyzed.

According to some embodiments of the invention, analyzing the level of CD157-expressing cells comprises analyzing the expression level and/or spatial distribution of CD157-expressing cells in the CNS of the subject.

It should be noted that the spatial distribution of CD157-expressing cells can vary during progression of the inflammatory disease or disorder in the CNS, and/or during treatment of the inflammatory disease or disorder in the CNS.

As mentioned, CD157-expressing cells can be identified, and their level in the CNS can be quantified using an agent which specifically binds a CD157 antigen on the surface of the cells using any immunological method in-vitro (e.g., immunohistochemistry, immunofluorescence, FACS, ELISA, and Western Blot) or in-vivo (e.g., using a detectable moiety attached to the agent (e.g., antibody, antibody mimetic or aptamer) for imaging in the subject, e.g., for Positron Emission Tomagraphy (PET), single-photon emission computed tomography (SPECT), Magnetic Resonance Imaging (MRI), bioluminescence and/or near-infrared (NIR) fluorescence).

The antibody can be attached directly to a detectable moiety, or indirectly such as by an affinity tag (a member of a binding pair) which is identifiable by a secondary binding moiety (or binding pair).

According to some embodiments of the invention, the antibody or fragment thereof is conjugated to a detection moiety.

Various types of detectable or reporter moieties may be conjugated to the agent (e.g., antibody or fragment thereof) of the invention. These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (e.g., contrast agents) detectable by Positron Emission Tomagraphy (PET), single-photon emission computed tomography (SPECT), Magnetic Resonance Imaging (MRI) bioluminescence and/or near-infrared (NIR).

For in-vivo detection the agent capable of binding CD157 (e.g., the anti-CD157 antibody, antibody fragment or mimetic thereof, aptamer) can be labeled with a radioactive moiety. The radioactive moiety is preferably inert and stable, and should have a preserved immunoreactivity.

Antibodies can be conjugated with a pair of radiometal and a chelator. One approach is to first modify the antibody with a chelator, and then radiolabeling the antibody.

For example, for Positron Emission Tomagraphy (PET) or single-photon emission computed tomography (SPECT) the antibody can be labeled with ⁸⁹Zr, ⁶⁴Cu, ⁶⁸Ga, ⁵²Mn, ⁸⁶Y ⁶⁶Ga, ⁴⁴Sc, and/or ¹⁸F as in [¹⁸F]AlF (reviewed in Marion Chomet et al., 2021; Bioconjug Chem. 32(7):1315-1330; “State of the Art in Radiolabeling of Antibodies with Common and Uncommon Radiometals for Preclinical and Clinical Immuno-PET”; which is fully incorporated herein by reference in its entirety).

For Magnetic Resonance Imaging (MRI) the antibody can be labeled with superparamagnetic iron oxide nanoparticles, gadolinium, or Manganese oxide nanoparticles.

The anti-CD157 agent can also be conjugated to bioluminescence and/or near-infrared (NIR) fluorescent probes or nanoparticles and be used for in-vivo applications.

Non-limiting examples of fluorophores and fluorescent nanoparticles include fluorophores (e.g. DyLight 680, Alexa Fluor 680, Cy5.5, IRFye680, DyLight 800, Cy-7, Cy-7.5, Alexa Fluor 790, IRDye800CW), indocyanine green (ICG), pafolacianine (Cytalux™), polymethine cyanine (e.g. IR-1040, IR-1048, IR-1051, IR-780, and IR-1061) or donor-acceptor-donor (D-A-D) architecture (e.g. CH1055).

In order to determine a statistically significant level of CD157-expressing cells in the subject in need thereof above a predetermined threshold the level is typically compared to control such as the level CD157-expressing cells in a control tissue or cells.

According to some embodiments of the invention, the control tissue corresponds to the brain area (region) which is analyzed in the brain of the subject in need thereof (e.g., the subject having the disease or disorder).

According to some embodiments of the invention, the control used in the method of diagnosing the inflammatory disease or disorder of the CNS is of subject(s) who are devoid of the inflammatory disease or disorder of the CNS (e.g., healthy subject(s)).

As used herein the phrase “a level of the CD157-expressing cells above a predetermined threshold” refers to at least about 10%, e.g., higher than about 20%, e.g., higher than about 30%, e.g., higher than about 40%, e.g., higher than about 50%, e.g., higher than about 60%, higher than about 70%, higher than about 80%, higher than about 90%, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to a predetermined threshold of the control.

According to an aspect of some embodiments of the invention there is provided a method of diagnosing and treating an inflammatory disease or disorder of the central nervous system (CNS) in a subject in need thereof, the method comprising:
(a) analyzing a level of CD157-expressing cells in the CNS of the subject, wherein a level of the CD157-expressing cells above a predetermined threshold is indicative of the inflammatory disease or disorder of the CNS, and
(b) administering to the subject a therapeutically effective amount of an agent capable of treating the inflammatory disease or disorder of the CNS in the subject,
thereby diagnosing and treating the neuroinflammatory disease or disorder in the subject.

According to some embodiments of the invention, the agent capable of treating the inflammatory disease or disorder of the CNS comprises an agent capable of binding CD157 on the CD157-expressing cells of the CNS, the agent capable of mediating a therapeutic effect.

The present inventors have further envisaged that presence of the CD157-expressing cells in the CNS can be used for identifying a tumor mass in the CNS, and thus can assist in removing the tumor mass during a surgery.

According to an aspect of some embodiments of the invention there is provided a method of treating a subject having a tumor mass in the central nervous system (CNS), the method comprising:
(a) analyzing a level of CD157-expressing cells in the CNS of the subject, wherein a level of the CD157-expressing cells above a predetermined threshold is indicative of presence of the tumor mass in the CNS, and
(b) resecting the tumor mass, wherein the resecting is guided by the CD157-expressing cells,
thereby treating the subject.

According to some embodiments of the invention, the method further comprising:
(c) repeating step (a) following resecting the tumor.

It should be noted that in a routine post-surgery MRI performed to detect the border(s) of a tumor mass it is often hard to distinguish between tumor boundaries and a post-surgery edema.

According to some embodiments of the invention, the method further comprises distinguishing between a tumor mass (identified by presence of CD157-expressing cells) or non-tumor edema (which devoid of CD157-expressing cells).

According to some embodiments of the invention, resecting is guided by monitoring presence of the CD157-expressing cells above a predetermined threshold using for example an imaging technique such as MRI.

According to some embodiments of the invention, the resecting is performed in real-time along with analyzing the level of CD157-expressing cells.

The present inventors have further envisaged that the level of CD157-expressing cells can be used to monitor the efficacy of treatment of the inflammatory disease or disorder of the central nervous system (CNS). For example, a decrease in the level of CD157-expressing cells following the treatment can indicate of the efficacy of the therapy. In addition, a change (e.g., reduction) in the spatial distribution of the CD157-expressing cells can indicate efficacy of treatment.

On the other hand, re-appearance of CD157-expressing cells in previous locations or in new locations within the CNS may suggest worsening of the disease.

According to an aspect of some embodiments of the invention there is provided a method of monitoring efficacy of a therapy to an inflammatory disease or disorder of the central nervous system (CNS) in a subject in need thereof, the method comprising analyzing a level of CD157-expressing cells in the CNS of the subject having been treated with an agent capable of treating the inflammatory disease or disorder of the CNS, and wherein a decrease in the level of CD157-expressing cells in the CNS of the subject from a predetermined threshold following the therapy is indicative of efficacy of the therapy, thereby monitoring efficacy of the therapy.

According to some embodiments of the invention, a decrease in the level of CD157-expressing cells in the CNS of the subject from a predetermined threshold refers to a decrease of least about 10%, about 20%, about 30%, about 40%, e.g., about 50%, e.g., about 60%, about 70%, about 80%, about 90%, about 95%, about 99% from the predetermined threshold as determined by a control.

According to some embodiments of the invention, the control used in the method of monitoring the efficacy of a therapy is of a subject who is diagnosed with the inflammatory disease or disorder of the CNS.

It should be noted that the control can be a reference expression data obtained from multiple control subjects (e.g., at least 2, 5, 10, 20 or more) who are diagnosed with the inflammatory disease or disorder of the CNS.

According to some embodiments of the invention, the predetermined threshold is the level of CD157-expressing cells in the subject prior to administering the therapy.

According to some embodiments of the invention, a decrease in the level of CD157-expressing cells in the CNS of the subject of least about 10%, about 20%, about 30%, about 40%, e.g., about 50%, e.g., about 60%, about 70%, about 80%, about 90%, about 95%, about 99% after administering the agent (the therapy) relative to the level of the CD157-expressing cells in the CNS of the subject prior to administering the agent is indicative of efficacy of the therapy (i.e., that the agent is efficient in treating the subject).

The exogenous polynucleotide encoding an expression product capable of alleviating at least one symptom of the inflammatory disease or disorder, wherein the exogenous polynucleotide is under the transcriptional control of a cis acting regulatory element active specifically in a CD157-expressing cell, can be part of a nucleic acid construct.

The nucleic acid construct is preferably suitable for mammalian cell expression. Such a nucleic acid construct includes a promoter sequence for directing transcription of polynucleotide sequence of interest in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

The vector may or may not include a eukaryotic replicon.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of the polynucleotide of interest since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, retrovirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

The agent which is capable of binding CD157 on CD157-expressing cells of the CNS of some embodiments of the invention, or the exogenous polynucleotide of some embodiments of the invention encoding an expression product capable of alleviating at least one symptom of the inflammatory disease or disorder, wherein the exogenous polynucleotide is under the transcriptional control of a cis acting regulatory element active specifically in a CD157-expressing cell, can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent which is capable of binding CD157 on CD157-expressing cells of the CNS of some embodiments of the invention, or the exogenous polynucleotide of some embodiments of the invention encoding an expression product capable of alleviating at least one symptom of the inflammatory disease or disorder, wherein the exogenous polynucleotide is under the transcriptional control of a cis acting regulatory element active specifically in a CD157-expressing cell, accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., the inflammatory disease or disorder of the central nervous system (CNS)) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in-vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, when a SEQ ID NO is expressed in a DNA sequence format (e.g., reciting T for thymine), it can refer to either a DNA sequence that corresponds to a nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Mice tdTomato^{fl/fl} (Ai9; RCL-tdT, Jax #007909), Cx3cr1^{creER-YFP} (B6.129P2(Cg)-Cx3cr1tm2.1(cre/ERT2)Litt/WganJ, Jax #021160), and C57BL/6J (B6/J, Jax #000664) mice were purchased from Jackson Laboratory (Bar Harbor, ME, USA). NOD-Cas9/GFP (Rosa26-LSL-Cas9 knockin on NOD, Jax #026431) were used. tdTomato^{fl/wt} Cx3cr1^{creER/wt} mice were generated by breeding Cx3cr1^{creER/creER} mice with tdTomato^{fl/fl} mice. F1 heterozygote mice were generated by breeding NOD mice and C57B1/6 mice. All animals were housed and maintained in laminar flow cabinets under specific pathogen-free (SPF) conditions in accordance with current regulations and standards of the Israel Ministry of Health. All animal protocols were approved by the Tel Aviv University Institutional Animal Care and Use Committee.

### EAE Induction and Tamoxifen Treatment

EAE was induced by immunization of female mice with myelin oligodendrocyte glycoprotein peptide (MOG₃₅₋₅₅) emulsified in complete Freund's adjuvant (Difco Laboratories) at a dose of 100 μg per mouse, followed by the administration of pertussis toxin (100 ng per mouse; List biological laboratories, Inc.) on Days 0 and 2 as previously described [L. Mayo et al., Nat Med (2014) 20(10):1147-56]. Clinical signs of EAE were assessed according to the following score: 0, no signs of disease; 1, loss of tone in the tail; 2, hind limb paresis; 3, hind limb paralysis; 4, tetraplegia; 5, moribund.

tdTomato^{fl/wt} Cx3cr1^{creER/wt} mice were treated with 100 mg/kg Tamoxifen (Sigma-Aldrich, #T5648) for three consecutive days. EAE was induced 7 weeks following tamoxifen treatment.

### Tissue Preparation and Immunohistochemistry

Animals were perfused transcardially with 10 ml of PBS. Spinal cords were removed, and post-fixed for 1 hour in 4% paraformaldehyde at room temperature. 10-μm-thick sections generated using cryostat. For immunofluorescence stain, sections were blocked in 5% goat or donkey serum, containing 0.3% Triton X-100 (Sigma-Aldrich), and incubated overnight at 4° C. with primary antibodies to the following: CD157 (Rabbit, 1:500, LSbio), GFP (chicken, 1:500, Abcam), IBA-1 (Rabbit, 1:200, Wako chemicals), IBA-1 (Goat, 1:250, Wako chemicals). The next day, sections were washed three times and incubated with an appropriate fluorophore-conjugated secondary antibody for 1 hour at room temperature: Goat anti-Rabbit AF488 (1:500, Invitrogen), Goat anti-Rabbit AF647 (1:500, Invitrogen), Goat anti-chicken AF488 (1:700, Abcam), Donkey anti-Goat AF594 (1:500, Invitrogen), Donkey anti-Rabbit AF647 (1:500, Invitrogen). Images were taken using a Leica SP8 confocal microscope. For histochemistry stain, sections were stained with Luxol® Fast blue for myelin. Images were taken using an Axio Zoom V16 microscope.

Isolation of Cells from Adult Mouse CNS and Flow Cytometry

Mononuclear cells were isolated from the CNS as previously described [Mayo L. et al., Nat Med (2014) 20: 1147-1156]. Cells were stained with CD11b (1:100, BioLegend or Invitrogen), CD45 (1:200, BioLegend, BioGems), CCR2 (1:200, R&D), Ly6C (1:100, BioLegend), CD157 (1:50, BD) or biotinylated anti-CD157 with fluorophore-conjugated streptavidin.

### Quantitative Polymerase Chain Reaction (PCR)

RNA was extracted with TRIzol (Invitrogen), cDNA was prepared (Thermo Scientific) and used for quantitative PCR with the following primers.

TABLE 2
qPCR sequences

| Gene | Forward primer | Reverse primer |
|---|---|---|
| Lag3 | CCAGGCCTCGATGAT | CAGCAGCGTACACTG |
| | TGCTA | TCAGA |
| | (SEQ ID NO: 1) | (SEQ ID NO: 2) |
| Chi11 | TACTACGAGATATGC | GGAAATCATCCAAAT |
| | GACTTC | CCAGTG |
| | (SEQ ID NO: 3) | (SEQ ID NO: 4) |
| Cc112 | TGTGATCTTCAGGAC | CATGAAGGTTCAAGG |
| | CATAC | ATGAAG |
| | (SEQ ID NO: 5) | (SEQ ID NO: 6) |
| Arg1 | CTCCAAGCCAAAGTC | AGGAGCTGTCATTAG |
| | CTTAGAG | GGACATC |
| | (SEQ ID NO: 7) | (SEQ ID NO: 8) |
| Nos2 | GGAGTGACGGCAAAC | TCGATGCACAACTGG |
| | ATGACT | GTGAAC |
| | (SEQ ID NO: 9) | (SEQ ID NO: 10) |
| Vegfa | CTGCTGTAACGATGA | GCTGTAGGAAGCTCA |
| | AGCCCTG | TCTCTCC |
| | (SEQ ID NO: 11) | (SEQ ID NO: 12) |
| P2ry12 | TACCCTACAGAAACA | GCTGAATCTGAAGGA |
| | CTCAAG | TATGAG |
| | (SEQ ID NO: 13) | (SEQ ID NO: 14) |
| Tmem119 | CCAGAGCTGGTTCCA | GGGAGTGACACAGAG |
| | TAG | TAG |
| | (SEQ ID NO: 15) | (SEQ ID NO: 16) |
| Csf1 | TAGAAAGGATTCTAT | CTCTTTGGTTGAGAG |
| | GCTGGG | TCTAAG |
| | (SEQ ID NO: 17) | (SEQ ID NO: 18) |

### Antibody Dependent Cell Death Assays In-Vitro

For Complement dependent cytotoxicity (CDC) assay, RAW264.7 cells were incubated for 30 minutes with anti-CD157 antibodies or isotype control. Next, cells were incubated with rabbit complement serum (Sigma-Aldrich) for 3 hours in 37° C.

For Antibody-dependent cellular cytotoxicity assay (ADCC), RAW264.7 cells were incubated for 30 minutes with anti-CD157 antibodies or isotype control, then freshly isolated NK cells (Stemcell, #19815) were added and incubated for 4 hours in 37° C.

Cell death was measured using LDH cytotoxicity detection kit (Roche).

### Antibody Treatment

For cell depletion experiments, adult female C57BL/6J wild-type (WT) mice received 100 μg anti-CD157 mAbs (BP3) or isotype control (Bio X Cell), daily, from day 7 post immunization. On day 10, blood samples were collected for FACS analysis of monocyte depletion. NOD-C57BL/6 F1 female mice received 50 μg anti-CD157 mAbs (BP3) or isotype control (Bio X Cell), every other day, from the beginning of the progressive phase of the disease (day 37).

### Peripheral Blood Analysis

Peripheral blood was obtained from the tail vein and collected into PBS with 5 mM EDTA. Red blood cells were lysed using Lysing Solution (BD) and the white blood cells were resuspended in FACS buffer for further staining. Peripheral blood cells were stained for CD45 (1:100, BioLegend), Ly6G (1:200, BioLegend), CD115 (1:200, BioGems), CD11b (1:200, BioLegend), Ly6C (1:200, BioLegend), CCR2 (1:100, R&D) and CD157 (1:50, BD).

### Bone Marrow Transplantation

4-6 weeks old C57BL/6J mice received three treatments of 30 mg/kg Busulfan (Sigma) every three days. Two days after the final treatment, bone marrow was isolated from Cx3cr1^{creER/creER} or C57BL/6J mice and 5×10⁶ cells were transplanted intravenously into the treated mice as previously described [K. Peake et al., J Vis Exp, (2015) e52553]. 10 weeks after transplantation, eYFP levels in the blood was assessed by FACS and mice were immunized for EAE.

### Immunoblot Analysis

Protein samples from the blood and CNS of naïve and EAE C56BL/6 mice were prepared in RIPA lysis buffer and separated by SDS-polyacrylamide gel electrophoresis. After transfer to nitrocellulose membranes, blots were probed separately against mouse CD157 (R&D) and Vinculin (Proteintech) as indicated. The secondary antibody used was horseradish peroxidase-linked anti-rabbit IgG (Cell Signaling Technology), and membranes were visualized using ImageQuant™ 800.

### RNAseq

RNA was extracted using Dynabeads™ mRNA DIRECT™ Purification Kit (Invitrogen). Sequencing: Sequencing Libraries were prepared using INCPM MARS-seq. Reads were sequenced on NUM_LANES lane(s) of an Illumina NovaSeq. The output was approximately 12 million reads per sample.

Bioinformatics: Poly-A/T stretches and Illumina adapters were trimmed from the reads using Cutadapt as previously described [Martin M., Cutadapt removes adapter sequences from high-throughput sequencing reads (2011) 17: 3]. Resulting reads shorter than 30 bp were discarded. Remaining reads were mapped onto 3′ UTR regions (1000 bases) of the M. musculus, mm10 genome according to Refseq annotations, using STAR [A. Dobin et al., STAR: ultrafast universal RNA-seq aligner. Bioinformatics (2012) 29: 15-21] with EndToEnd option and outFilterMismatchNoverLmax set to 0.05. Deduplication was carried out by flagging all reads that were mapped to the same gene and had the same UMI. Counts for each gene were quantified using htseq-count [Anders S. et al., HTSeq—a Python framework to work with high-throughput sequencing data. Bioinformatics (2014) 31: 166-169], using the gtf above and corrected for UMI saturation. Differentially expressed genes were identified using DESeq2 [Love M. I. et al., Moderated estimation of fold change and dispersion for RNA-seq data with DESeq2. Genome Biology (2014) 15:550] with the betaPrior, cooksCutoff and independentFiltering parameters set to False. Raw P values were adjusted for multiple testing using the procedure of Benjamini and Hochberg. Pipeline was run using snakemake [J. Köster and S. Rahmann, Snakemake—a scalable bioinformatics workflow engine. Bioinformatics (2012) 28: 2520-2522].

### Statistical Analysis

Statistical data analysis was performed with Prism software version 8.3 (GraphPad Software). For comparisons of two groups, two-tailed Student's t-test was used. Comparisons of multiple groups were made using one-way or 2-way ANOVA, as described in the figure legends. Analysis of EAE clinical scores was corrected for repeated measures and subjected to linear regression analysis (95% confidence interval) for the analysis of the treatment effect on disease progression. Data represent mean±standard error of the mean. P<0.05 was considered significant.

### Example 1

### CD157 Expression is Upregulated in the CNS During Neuroinflammation

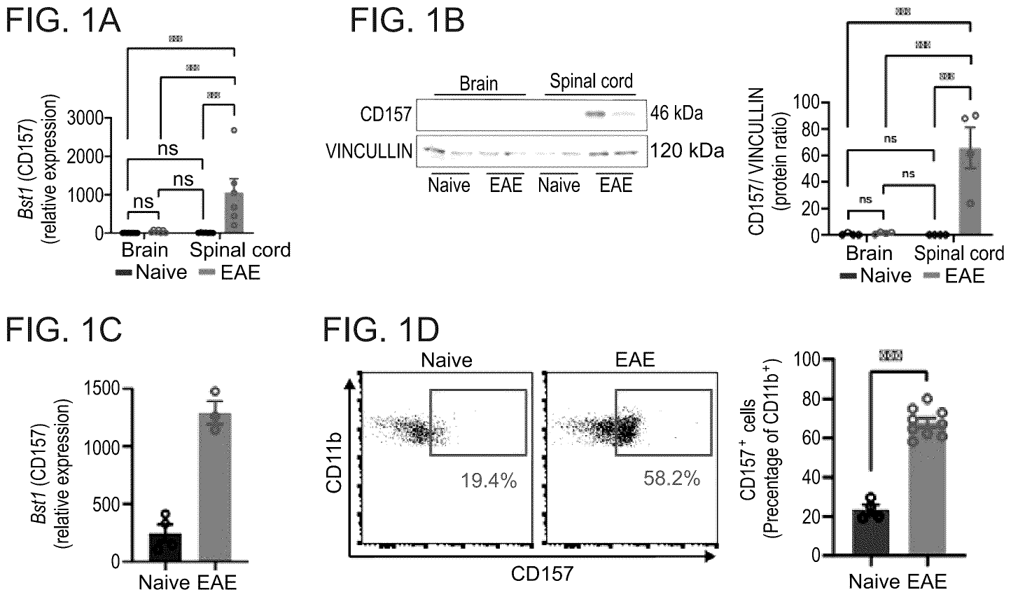

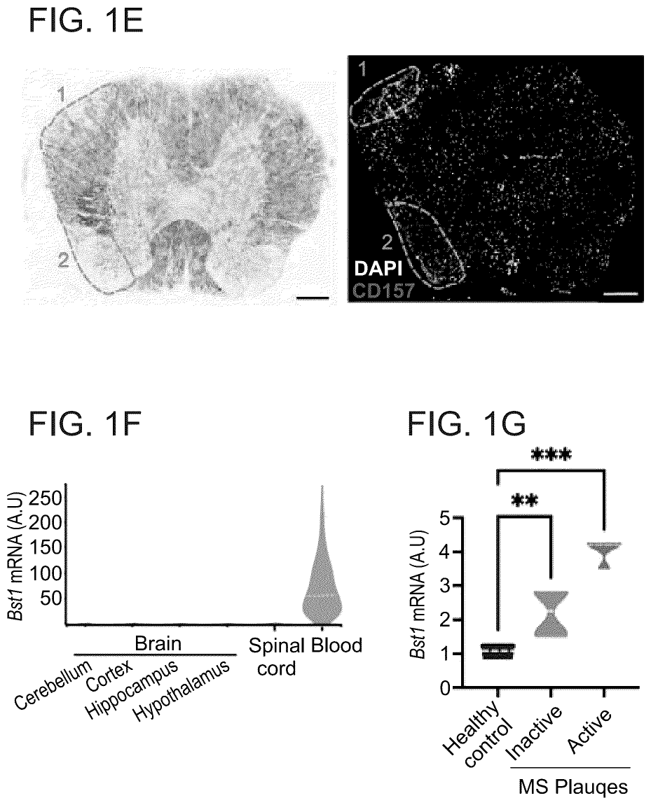

To study whether CD157 is a potential marker for infiltrating cells in the CNS, the expression of CD157 in the blood and CNS of naïve and EAE mice was studied. Blood samples, brain, and spinal cord were collected from C57BL/6 mice immunized with myelin oligodendrocyte glycoprotein peptide (MOG₃₅₋₅₅), at the peak of the disease. It was uncovered that CD157 (Bst1) expression is significantly elevated in the blood during EAE, and while CD157 expression could not be detected in the naïve CNS, there was a significant elevation during EAE (FIGS. 1A-D). Furthermore, from histopathological analysis of sections obtained from EAE spinal cords, it was uncovered that CD157⁺ cells are localized in the lesion area, shown by Luxol® Fast blue and immunofluorescence staining of sister slides (FIG. 1E). Similarly, BST1 expression was not detected in the brain or spinal cord tissue of normal patients (FIG. 1F), but was found in MS plaques (FIG. 1G). Taken together, this data suggests that CD157 is expressed in the CNS by inflammatory immune cells during EAE and MS.

### Example 2

### CD157 Expression in the CNS is Confined to Infiltrating Monocytes

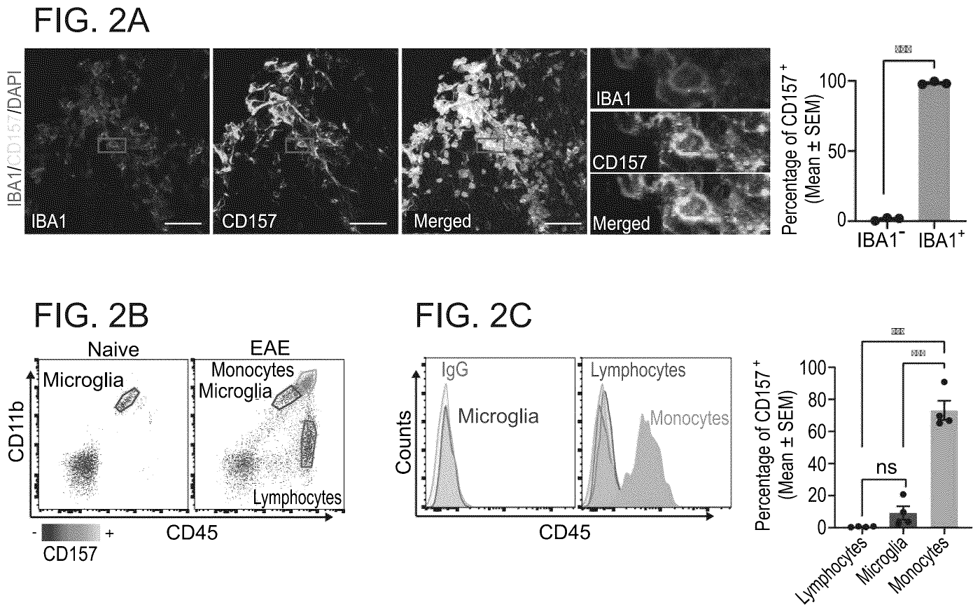

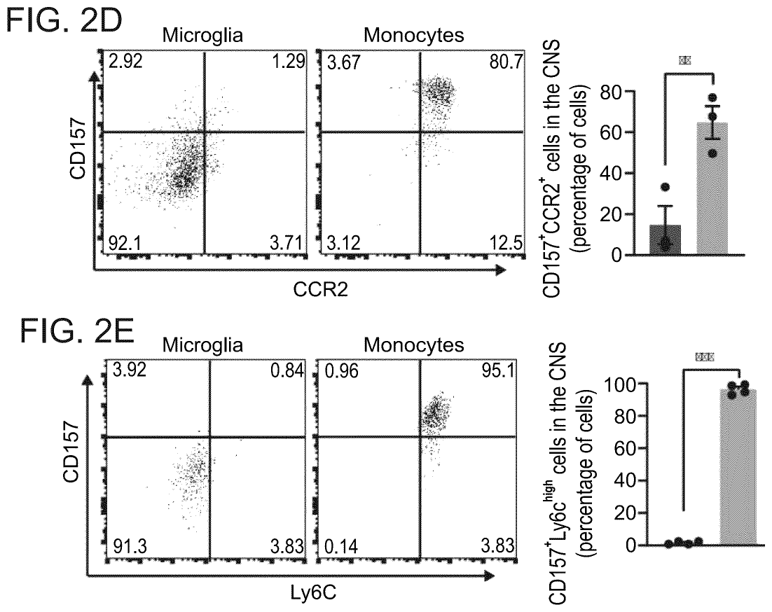

To investigate the origin of CD157 during neuroinflammation, the co-localization of CD157 with the microglial and monocyte marker ionized calcium-binding adaptor molecule 1 (IBA1) was studied in lumbar spinal cord sections from EAE mice. A 98.4±0.45% of co-localization of CD157 staining with IBA1 expression (FIG. 2A) was found, suggesting that CD157⁺ cells in the CNS are infiltrating monocytes or resident microglial cells.

Next, cells from the CNS of naïve and EAE mice were isolated and examined for the expression of CD157 by flow cytometry. From CD11b and CD45 gating, it was found that CD157 is expressed by CD11b⁺CD45^{high} cells (i.e. monocytes) and not by CD11b⁺CD45^{low} (i.e. microglial cells), or other cell populations (FIGS. 2B and 2C). Further analysis of CD11b⁺CD45^{low} and CD11b⁺CD45^{high} cells in the CNS revealed that monocytes are CD157⁺CCR2⁺ (61.18±9.78) and CD157⁺Ly6c^{high} (60.16±9.27), compared to the microglial population (9.39±3.44 and 1.58±0.43, respectively) (FIGS. 2D and 2E). Thus, CD157⁺ cells are also CD45^{high} CCR2⁺Ly6c^{high}.

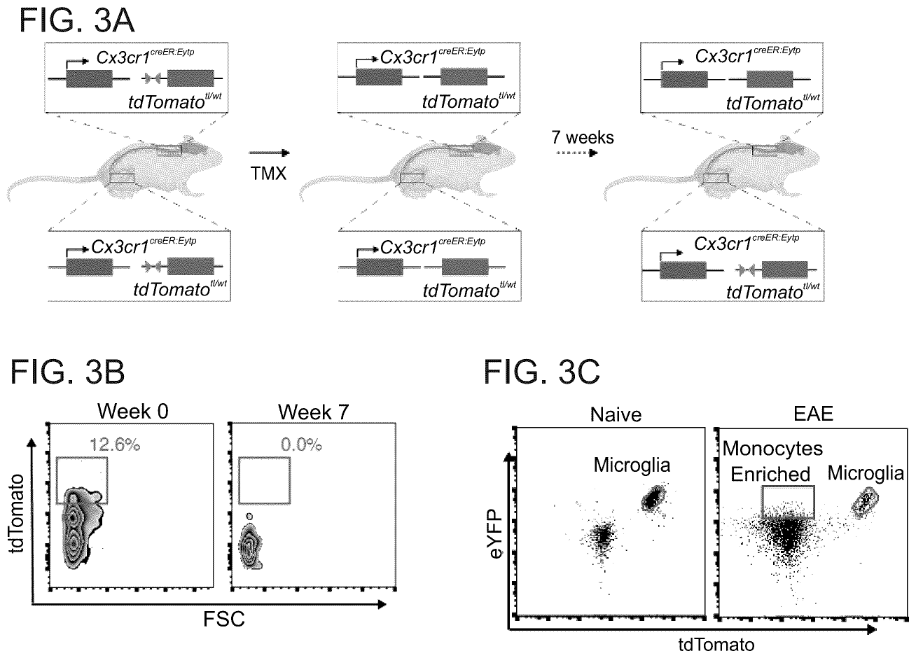

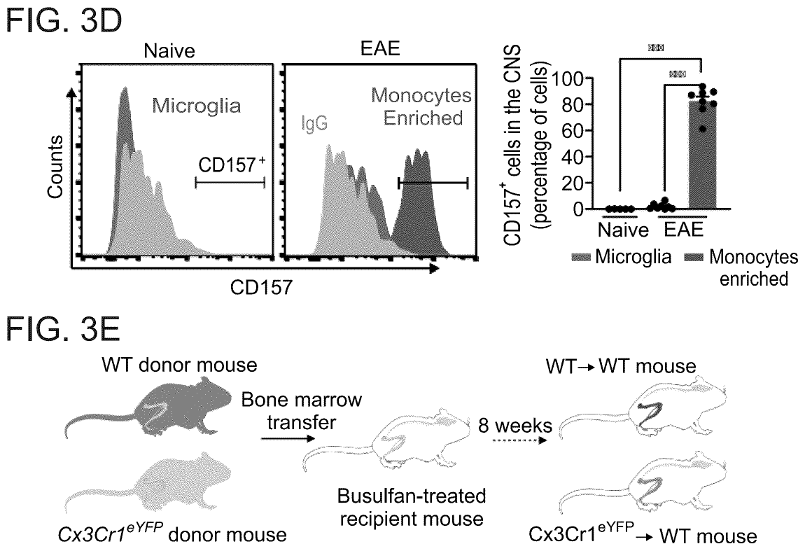

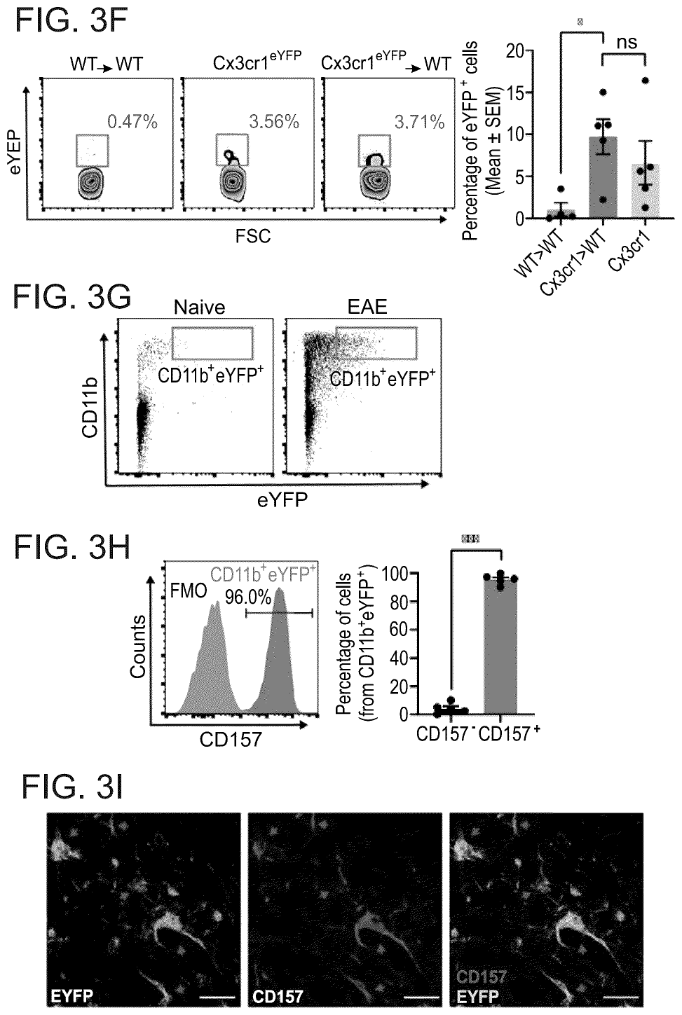

CD45 levels of expression can vary in different inflammatory states, and therefore is not an optimal method to distinguish infiltrating monocytes from resident microglial cells during neuroinflammation. To study the expression of CD157 by resident microglial cells, a fate mapping model was generated (FIG. 3A). tdTomato^{fl/fl} mice were crossed with Cx3cr1^{creER:Eyfp} to create Cx3cr1^{creER:Eyfp} tdTomato^{fl/wt} hybrid. Following tamoxifen (TMX) treatment, Cx3cr1 expressing cells, i.e., monocytes and microglial cells, express tdTomato. However, since monocytes are a short living population, up to 7 weeks after tamoxifen treatment, tdTomato⁺ monocytes were replaced by new tdTomato⁻ monocytes from the bone marrow, while the microglial cells remained tdTomato⁺ even after a prolonged period of time [S. Yona et al., Immunity (2013) 38: 79-91]. From flow cytometry analysis of blood samples collected after TMX treatment, tdTomato expression by CD45⁺ cells was found (10.6%±1.71, left panel in FIG. 3B), which was reversed 7 weeks after treatment (0.24%±0.15, right panel in FIG. 3B). Cx3cr1^{creER:Eyfp} tdTomato^{fl/wt} mice were immunized for EAE at this time point. By flow analysis of the cell populations in the CNS, it was uncovered that eYFP⁺tdT⁺ cells (both naïve and activated microglia) do not express CD157 (0.32%±0.14 and 2.67±0.67, respectively, FIGS. 3C), compared to eYFP⁺tdT⁻ cells (monocyte enriched population) which expressed high levels of CD157 (81.59±3.00) (FIGS. 3C-E).

While the microglia population is a self-renewed population, new monocytes are constantly generated from bone marrow-resident precursor cells. To investigate the expression of CD157 by infiltrating monocytes, a bone-marrow transplantation model was created, marking specifically bone marrow-derived monocytes (FIG. 3E).

To this end, following chemotherapy (e.g. with Busulfan) treatment, C57BL/6 WT mice received bone marrow transplantation from C57BL/6 WT or Cx3cr1^{creER:Eyfp} mice to replace their original bone marrow. Transplantation was evaluated by eYFP levels in the blood of transplanted mice (FIG. 3F). Next, transplanted mice were immunized for EAE and cellular infiltration to the CNS of these mice was analyzed. As evident from the results, it was found that CD11b⁺eYFP⁺ cells are only present in the CNS during EAE, and not in naïve, indicating these are infiltrating cells (FIGS. 3G-H). Furthermore, it was found that CD11b⁺eYFP⁺ cells are also CD157⁺ (93.06±1.78, FIG. 3H). Correspondingly, immunofluorescence staining showed that eYFP⁺ cells are also CD157⁺ (95.66±0.84, FIG. 3I). Taken together, this data demonstrate that CD157 can be used as a novel marker to identify infiltrating monocytes during neuroinflammation and is not expressed by resident microglial cells.

### Example 3

Anti-CD157 mAbs Mediates Cell Death and Ameliorates Disease Development

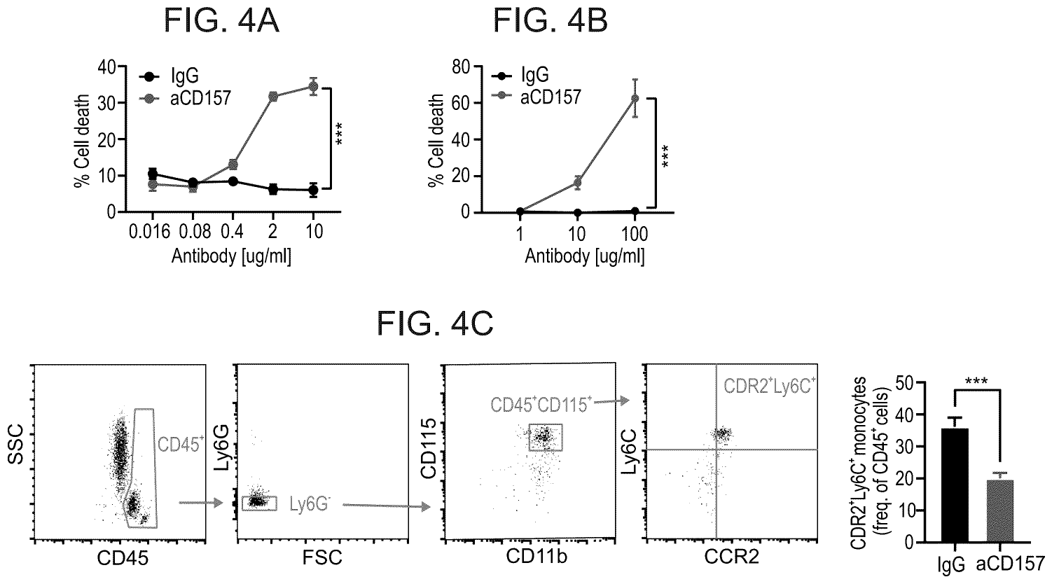

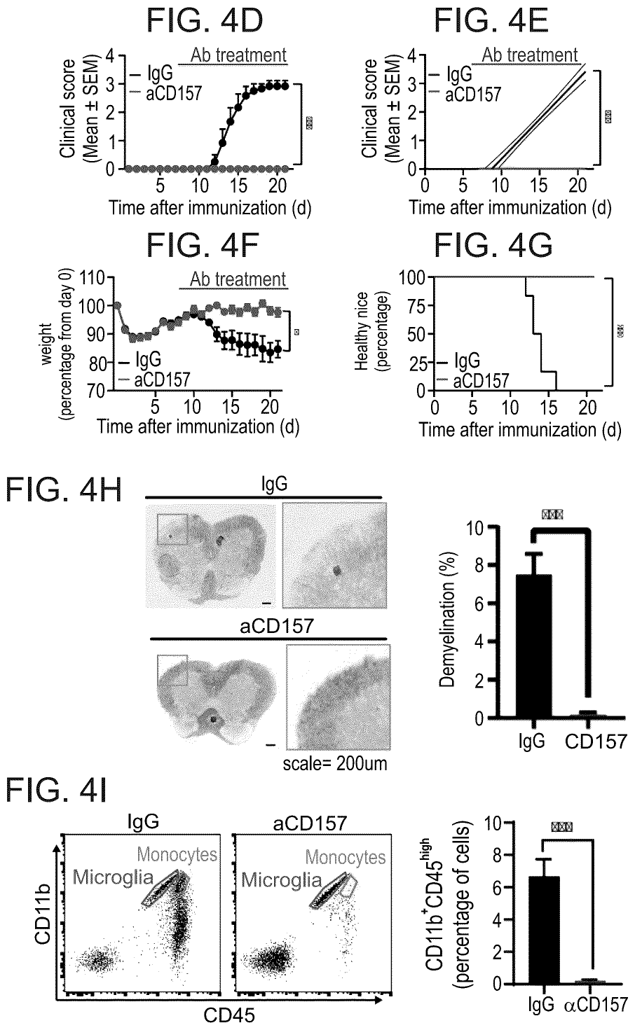

Targeting infiltrating monocytes during neuroinflammation is a major unmet need. To this end, anti-CD157 mAbs were generated which are capable of mediating in-vitro complement-dependent cytotoxicity (CDC) (FIG. 4A) and antibody-dependent cellular cytotoxicity (ADCC) (FIG. 4B) when tested on RAW264.7 murine macrophage cell line. As evident from the results, it was uncovered that in-vivo administration of anti-CD157 mAbs significantly depleted CCR2⁺ly6C⁺ monocytes, compared to isotype control (FIG. 4C). Daily administration of 100 μg anti-CD157 mAbs, from day 7 post-immunization, prevented EAE development and prevented weight loss in EAE mice (FIGS. 4D-G). Further analysis studied the effect of antibody treatment on suppression of disease progression in terms of demyelination, shown by Luxol® Fast Blue stain, and cellular infiltration (FIG. 4H). Next, the effect of anti-CD157 administration on cell infiltration in the CNS was investigated. As evident from the results, a significant reduction in monocyte infiltration was evident in the CNS of mice treated with anti-CD157 mAbs (FIG. 4I).

### Example 4

### CD157 is a Marker for Infiltrating Monocytes in the Progressive Model of EAE

Different mouse strains present different clinical forms of EAE and model different disease aspects. For example, immunization of C57BL/6 with MOG₃₅₋₅₅ results in an acute-monophasic attack, while immunization of non-obese diabetic (NOD) or NOD C57BL/6 hybrid mice with MOG₃₅₋₅₅ results in an acute phase, followed by a progressive disease form presenting irreversible accumulation of neurological impairment. In the acute-monophasic model of EAE, the adaptive immune system plays a pivotal in disease initiation and progression, while in the progressive model, the progressive phase is mainly orchestrated by the innate immune system, e.g., monocytes [L. Mayo et al., Brain (2016) 139: 1939-1957]. Thus, finding a marker for infiltrating monocytes during the progressive phase of the disease is a major unmet need.

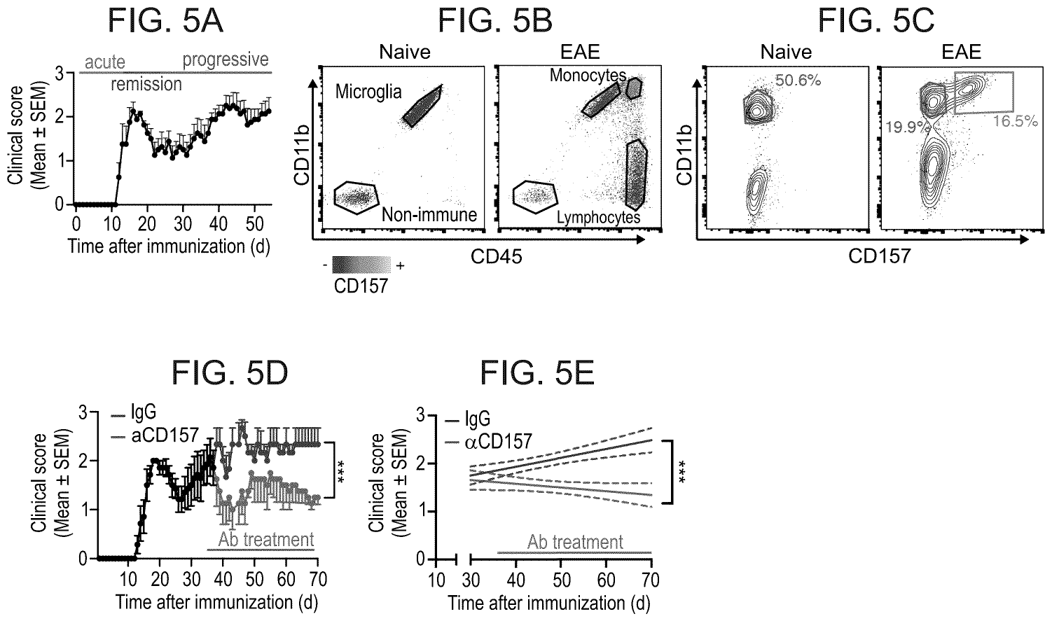

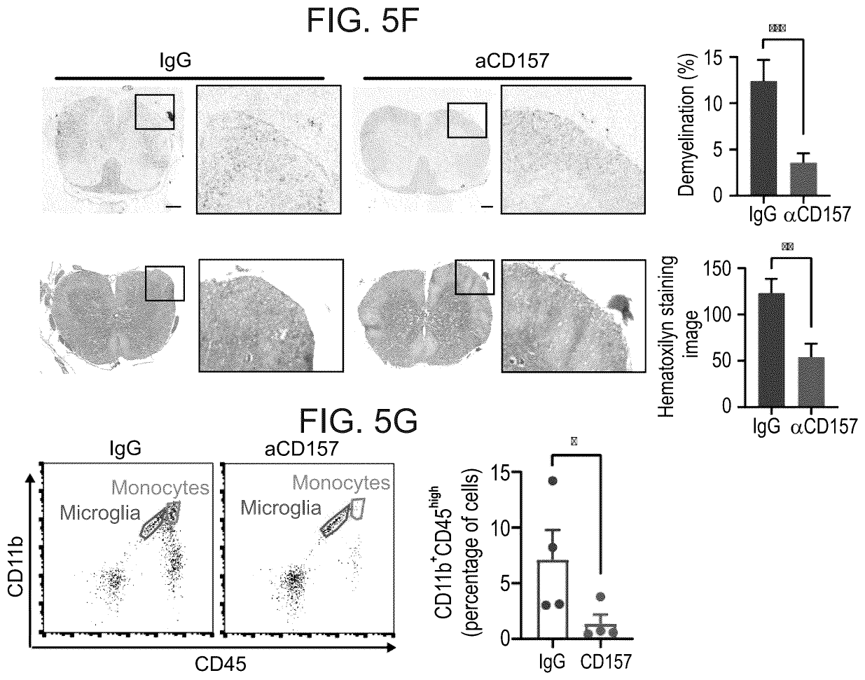

To investigate the role of CD157⁺ cells in the progressive form of EAE, C57BL/6 and NOD F1 hybrid mice were immunized with MOG₃₅₋₅₅ and CD157 expression was analyzed in the CNS at the progressive phase of the disease (FIG. 5A). As evident from the results, CD11b⁺CD157⁺ cells are present only in the inflamed CNS (FIG. 5B) and CD157 is expressed by infiltrating monocytes (CD11b⁺CD45^{high} cells), and not by other cell populations (FIG. 5C). Thus, CD157 expression can be used to identify infiltrating monocytes also at the progressive form of EAE.

To further investigate the role of CD157+ monocytes in the progressive phase of NOD EAE the mice were treated with anti-CD157 or isotype-control mAbs to eliminate the cells. Daily mAb administration (50 μg pre mouse), initiated 40 days after EAE induction resulted in suppression of disease progression in terms of clinical score, demyelination and axonal loss (FIGS. 5D-F), and significantly decrease monocytes recruitment to the CNS (FIG. 5G).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating an inflammatory disease or disorder of the central nervous system (CNS) in a subject in need thereof, the method comprising administering to the subject a therapeutically effective amount of an agent capable of binding CD157 on CD157-expressing cells of the CNS, said agent capable of mediating a therapeutic effect, thereby treating the inflammatory disease or disorder of the CNS in the subject.

2. The method of claim 1, wherein said agent comprises an exogenous polynucleotide encoding an expression product capable of alleviating at least one symptom of the inflammatory disease or disorder, wherein said exogenous polynucleotide is under the transcriptional control of a cis acting regulatory element active specifically in a CD157-expressing cell, optionally, wherein said cis acting regulatory element is a promoter, optionally, wherein said promoter comprises a bst1 promoter, optionally, wherein said exogenous polynucleotide is encapsulated in a particle, optionally, wherein said particle is a viral particle, thereby treating the inflammatory disease or disorder of the CNS in the subject.

3. A method of diagnosing and treating an inflammatory disease or disorder of the central nervous system (CNS) in a subject in need thereof, the method comprising:
(a) analyzing a level of CD157-expressing cells in the CNS of the subject, wherein said CD157-expressing cells comprise myeloid cells wherein a level of said CD157-expressing cells above a predetermined threshold is indicative of said inflammatory disease or disorder of the CNS, and
(b) administering to the subject a therapeutically effective amount of an agent capable of treating the inflammatory disease or disorder of the CNS in the subject, optionally, wherein said agent capable of treating the inflammatory disease or disorder of the CNS comprises an agent capable of binding CD157 on said CD157-expressing cells of said CNS, said agent capable of mediating a therapeutic effect, and optionally, wherein said agent capable of treating the inflammatory disease or disorder of the CNS is selected from the group consisting of an anti-inflammatory drug, an immunosuppressant drug, an immunomodulatory drug, a neuroprotective drug, a cognitive enhancing drug, a remyelination agent and an antitumor agent, optionally, wherein said antitumor agent is a localized cancer targeting therapy,
thereby diagnosing and treating the neuroinflammatory disease or disorder in the subject.

4. The method of claim 3, further comprising resecting said tumor mass, wherein said resecting is guided by said CD157-expressing cells.

5. The method of claim 3, wherein said analyzing said level of CD157-expressing cells in the CNS is of a subject having been treated with an agent capable of treating the inflammatory disease or disorder of the CNS, and wherein a decrease in the level of CD157-expressing cells in the CNS of the subject from a predetermined threshold following the therapy is indicative of efficacy of the therapy.

6. The method of claim 1, wherein the agent is an antibody or fragment thereof, optionally, wherein said antibody comprises an anti-CD157 monoclonal antibody or fragment thereof, optionally, wherein said antibody or fragment thereof is conjugated to a therapeutic moiety, optionally, wherein said therapeutic moiety is an anti-inflammatory cytokine, optionally, wherein said therapeutic moiety is an agent capable of downregulating an expression of a protein upregulated in the neuroinflammatory disease or disorder, optionally, wherein said therapeutic moiety is a chemotherapeutic agent, optionally, wherein said antibody or fragment thereof is conjugated to a detection moiety.

7. The method of claim 1, wherein said agent is a small molecule.

8. The method of claim 2, wherein said expression product comprises a protein.

9. The method of claim 8, wherein said protein is selected from the group consisting of an anti-inflammatory protein, a Neuronal survival factor, and a factor supporting remyelination.

10. The method of claim 1, wherein said inflammatory disease or disorder of the CNS is a cancerous disease.

11. The method of claim 10, wherein the cancerous disease is selected from the group consisting of Acoustic neuroma, Astrocytoma, Choroid plexus carcinoma, Craniopharyngioma, Embryonal tumor, Ependymoma, Glioblastoma, Glioma, Medulloblastoma, Meningioma, Oligodendroglioma, Pediatric brain tumor, Pineoblastoma, Pituitary tumor and Brain metastasis.

12. The method of claim 1, wherein said inflammatory disease or disorder of the CNS is selected from the group consisting of Multiple Sclerosis (MS), Acute disseminated encephalomyelitis (ADEM), Acute Optic Neuritis (AON), Transverse Myelitis, Tauopathy, Neuromyelitis Optica (NMO), Autoimmune encephalitis, Viral encephalitis, Rasmussen's syndrome, Acute necrotizing encephalopathy of childhood (ANEC), Opsoclonus-myoclonus ataxia syndrome (OMAS), Parkinson's disease (PD), Alzheimer's disease (AD), Niemann's disease, Huntington's disease, Creutzfeldt-Jakob disease, Traumatic brain injury, Stroke, Spinal cord injury, neuroblastoma, Amyotrophic lateral sclerosis (ALS) and Spinal Muscular Atrophy (SMA).

13. The method of claim 1, wherein said inflammatory disease or disorder of the CNS is Multiple Sclerosis (MS), optionally, wherein said Multiple Sclerosis (MS) is primary progressive multiple sclerosis or secondary progressive multiple sclerosis, optionally, wherein said Multiple Sclerosis (MS) is relapsing remitting multiple sclerosis.

14. The method of claim 1, wherein said CD157-expressing cells comprise monocytes, optionally, wherein said monocytes comprise CNS-infiltrating monocytes.

15. The method of claim 14, wherein said monocytes express at least one of CD11b, CD45, CCR2, CX3CR1, and CD14, optionally, wherein said monocytes have a CD157⁺CD45⁺CCR2⁺, CD157⁺CD14⁺CD16⁺, CD157⁺CD14⁺CD16⁻, CD157⁺CX3CR1⁺ or a CD157⁺CD45⁺CD11b⁺ signature.

16. A chimeric polynucleotide comprising a nucleic acid sequence encoding an expression product capable of alleviating at least one symptom of an inflammatory disease or disorder and another nucleic acid sequence comprising a cis acting regulatory element specifically active in a CD157-expressing cell.

17. The chimeric polynucleotide of claim 16, wherein said cis acting regulatory element is a promoter, optionally, wherein said promoter comprises a bst1 promoter.

18. A composition of matter comprising the chimeric polynucleotide of claim 16, and a particle encapsulating or attached to said chimeric polynucleotide.

19. The composition of matter of claim 18, wherein said particle is a viral particle.

20. The chimeric polynucleotide of claim 16, wherein said expression product comprises a protein, optionally, wherein said protein is selected from the group consisting of an anti-inflammatory protein, a Neuronal survival factor, and a factor supporting remyelination.


SEQUENCE LISTING (39 sequences)

SEQ ID NO: 1 - DNA, 20 bp, synthetic construct
  misc_feature 1..20: note = Single strand DNA oligonucleotide
  source 1..20: mol_type = other DNA
ccaggcctcgatgattgcta

SEQ ID NO: 2 - DNA, 20 bp, synthetic construct
  misc_feature 1..20: note = Single strand DNA oligonucleotide
  source 1..20: mol_type = other DNA
cagcagcgtacactgtcaga

SEQ ID NO: 3 - DNA, 21 bp, synthetic construct
  misc_feature 1..21: note = Single strand DNA oligonucleotide
  source 1..21: mol_type = other DNA
tactacgagatatgcgacttc

SEQ ID NO: 4 - DNA, 21 bp, synthetic construct
  misc_feature 1..21: note = Single strand DNA oligonucleotide
  source 1..21: mol_type = other DNA
ggaaatcatccaaatccagtg

SEQ ID NO: 5 - DNA, 20 bp, synthetic construct
  misc_feature 1..20: note = Single strand DNA oligonucleotide
  source 1..20: mol_type = other DNA
tgtgatcttcaggaccatac

SEQ ID NO: 6 - DNA, 21 bp, synthetic construct
  misc_feature 1..21: note = Single strand DNA oligonucleotide
  source 1..21: mol_type = other DNA
catgaaggttcaaggatgaag

SEQ ID NO: 7 - DNA, 22 bp, synthetic construct
  misc_feature 1..22: note = Single strand DNA oligonucleotide
  source 1..22: mol_type = other DNA
ctccaagccaaagtccttagag

SEQ ID NO: 8 - DNA, 22 bp, synthetic construct
  misc_feature 1..22: note = Single strand DNA oligonucleotide
  source 1..22: mol_type = other DNA
aggagctgtcattagggacatc

SEQ ID NO: 9 - DNA, 21 bp, synthetic construct
  misc_feature 1..21: note = Single strand DNA oligonucleotide
  source 1..21: mol_type = other DNA
ggagtgacggcaaacatgact

SEQ ID NO: 10 - DNA, 21 bp, synthetic construct
  misc_feature 1..21: note = Single strand DNA oligonucleotide
  source 1..21: mol_type = other DNA
tcgatgcacaactgggtgaac

SEQ ID NO: 11 - DNA, 22 bp, synthetic construct
  misc_feature 1..22: note = Single strand DNA oligonucleotide
  source 1..22: mol_type = other DNA
ctgctgtaacgatgaagccctg

SEQ ID NO: 12 - DNA, 22 bp, synthetic construct
  misc_feature 1..22: note = Single strand DNA oligonucleotide
  source 1..22: mol_type = other DNA
gctgtaggaagctcatctctcc

SEQ ID NO: 13 - DNA, 21 bp, synthetic construct
  misc_feature 1..21: note = Single strand DNA oligonucleotide
  source 1..21: mol_type = other DNA
taccctacagaaacactcaag

SEQ ID NO: 14 - DNA, 21 bp, synthetic construct
  misc_feature 1..21: note = Single strand DNA oligonucleotide
  source 1..21: mol_type = other DNA
gctgaatctgaaggatatgag

SEQ ID NO: 15 - DNA, 18 bp, synthetic construct
  misc_feature 1..18: note = Single strand DNA oligonucleotide
  source 1..18: mol_type = other DNA
ccagagctggttccatag

SEQ ID NO: 16 - DNA, 18 bp, synthetic construct
  misc_feature 1..18: note = Single strand DNA oligonucleotide
  source 1..18: mol_type = other DNA
gggagtgacacagagtag

SEQ ID NO: 17 - DNA, 21 bp, synthetic construct
  misc_feature 1..21: note = Single strand DNA oligonucleotide
  source 1..21: mol_type = other DNA
tagaaaggattctatgctggg

SEQ ID NO: 18 - DNA, 21 bp, synthetic construct
  misc_feature 1..21: note = Single strand DNA oligonucleotide
  source 1..21: mol_type = other DNA
ctctttggttgagagtctaag

SEQ ID NO: 19 - DNA, 1979 bp, Homo sapiens
  source 1..1979: mol_type = genomic DNA
atcagtttgcggaaccgccttggtagaaggagagaaggggagtggaggaagcacgggactggagggaccaaagttccccgatggcggcccaggggtgcgcggcatcgcggctgctccagctgctgctgcagcttctgcttctactgttgctgctggcggcgggcggggcgcgcgcgcggtggcgcggggagggcaccagcgcacacttgcgggacatcttcctgggccgctgcgccgagtaccgcgcactgctgagtcccgagcagcggaacaagaactgcacagccatctgggaagcctttaaagtggcgctggacaaggatccctgctccgtgctgccctcagactatgacctttttattaacttgtccaggcactctattcccagagataagtccctgttctgggaaaatagccacctccttgttaacagctttgcagacaacacccgtcgttttatgcccctgagcgatgttctgtatggcagggttgcagatttcttgagctggtgtcgacagaaaaatgactctggactcgattaccaatcctgccctacatcagaagactgtgaaaataatcctgtggattccttttggaaaagggcatccatccagtattccaaggatagttctggggtgatccacgtcatgctgaatggttcagagccaacaggagcctatcccatcaaaggtttttttgcagattatgaaattccaaacctccagaaggaaaaaattacacgaatcgagatctgggttatgcatgaaattgggggacccaatgtggaatcctgcggggaaggcagcatgaaagtcctggaaaagaggctgaaggacatggggttccagtacagctgtattaatgattaccgaccagtgaagctcttacagtgcgtggaccacagcacccatcctgactgtgccttaaagtcggcagcagccgctactcaaagaaaagccccaagtctttatacagaacaaagggcgggtcttatcattcccctctttctggtgctggcttccaggactcaactgtaactggaaactgtgttgctctaaccctcctccagccctgcagcctccccttgcagtcatcattcgtgttctgtgtataccaaatgattctgttatctaaagaagctttttgctgggaaaacgatgtcctgaaaatggtatttcaatgaggcatatgttcaggatttcagaaacaagaagttagttctatttagcaggttaaaaaatgctgcattagaattaaagcaagttattttcttatttgtataatgacacaaagcattgggagtcagactgcttgtatattatcaaacattttaagagaattctaataaagctgtattttacatcatctatattttcctatatctccagcagcacggacactgcatgcttgttgattgcttaagacatatgtatactatataaatgtatgcatatatggtctgcaaagttttcatttttcatttatttggtataaagatgttcagttaatggttcctttttgttttgagagacagagtttcgctcctttgcccaggctggagtgaggtggggagatctcagctcactgcaacctctgcttccaggattcaagcgattctcctgcctcagcctcccaagtagctgggattgcaggtgcctgccaccatgtccagctaatttttgtatttttagtagagatggggtttcaccatgttggccaggctagtctcgaattcctgacctcaggtgatccacccacctcagcctcccaaagtgctaggattacaggcgtgagccaccacatccagccataatagttacttataatgagggaaaaacccaaaatatccagtaatatttacatatttactgaaactgtttggaacttgtattagtttgttcttgcattgctataaagaactgcccgagactgggtaatttacaaaaataaaaataaaaaagaggtttaattgactcaca

SEQ ID NO: 20 - protein, 318 aa, Homo sapiens
MAAQGCAASRLLQLLLQLLLLLLLLAAGGARARWRGEGTSAHLRDIFLGRCAEYRALLSPEQRNKNCTAIWEAFKVALDKDPCSVLPSDYDLFINLSRHSIPRDKSLFWENSHLLVNSFADNTRRFMPLSDVLYGRVADFLSWCRQKNDSGLDYQSCPTSEDCENNPVDSFWKRASIQYSKDSSGVIHVMLNGSEPTGAYPIKGFFADYEIPNLQKEKITRIEIWVMHEIGGPNVESCGEGSMKVLEKRLKDMGFQYSCINDYRPVKLLQCVDHSTHPDCALKSAAAATQRKAPSLYTEQRAGLIIPLFLVLASRTQL

SEQ ID NO: 21 - protein, 216 aa, Pseudomonas aeruginosa
AEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 22 - DNA, 651 bp, Pseudomonas aeruginosa
  source 1..651: mol_type = genomic DNA
gcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggaccttgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccacgcaacgtcggcggcgacctcgccccgtccagcatccccgaccaggaacaggcgatcagcgccctgccggactacgccagccagcccggcaaaccgtcgcgcgaggacctgaagtaa

SEQ ID NO: 23 - protein, 536 aa, Corynebacterium diphtheriae
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 24 - DNA, 1611 bp, Corynebacterium diphtheriae
  source 1..1611: mol_type = genomic DNA
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcatctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcatcggattccataggcgttcttgggtaccagaaaacagtagatcacaccaaggttaattctaagctatcgctattttttgaaatcaaaagctga

SEQ ID NO: 25 - protein, 127 aa, synthetic construct
  REGION 1..127: note = interleukin 2 (IL-2), partial [synthetic construct]
TKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - DNA, 698 bp, synthetic construct
  misc_feature 1..698: note = Synthetic gene for interleukin 2 (IL-2) (partial)
  source 1..698: mol_type = other DNA
  misc_difference 4: note = n is a, c, g, or t
gggngggggacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaaaaaaaaaaaaaccccccccc

SEQ ID NO: 27 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 28 - DNA, 1311 bp, Homo sapiens
  source 1..1311: mol_type = genomic DNA
gtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaaccccccctcccactgtaggccactggatggtcatttggcatctccgtatatgtgctctggctcctcagctgagagagaaaaaaataaactgtatttggctgc

SEQ ID NO: 29 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 30 - DNA, 2406 bp, Homo sapiens
  source 1..2406: mol_type = genomic DNA
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - protein, 153 aa, Homo sapiens [sequence omitted]

SEQ ID NO: 32 - DNA, 921 bp, Homo sapiens
  source 1..921: mol_type = genomic DNA
ttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 33 - protein, 365 aa, Homo sapiens [sequence omitted]

SEQ ID NO: 34 - DNA, 4000 bp, Homo sapiens
  source 1..4000: mol_type = genomic DNA
aagcttactctctggcaccaaactccatgggatgatttttccttcctagaagagtccaggtggacaggtaaggagtgggagtcagggagtccagttccagggacagagattacgggataaaaagtgaaaggagagggacggggcccatgccgagggtttctcccttgtttctcagacagctcttgggccaagactcagggagacattgagacagagcgcttggcacagaagcagaggggtcagggcgaagtccagggccccaggcgttggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggtgagtgcggggtcgggagggaaacggcctctgtggggagaagcaacgggccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaatttccacggaataggagattatcccaggtgcctgtgtccaggctggtgtctgggttctgtgctcccttccccatcccaggtgtcctgtccattctcaagatagccacatgtgtgctggaggagtgtcccatgacagatcgaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcttttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttgg

SEQ ID NO: 35 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 36 - DNA, 1601 bp, Homo sapiens
  source 1..1601: mol_type = genomic DNA
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 37 - protein, 576 aa, Ricinus communis
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 38 - DNA, 12150 bp, Ricinus communis
  source 1..12150: mol_type = genomic DNA
atatttcacgaactgatacaatatagcaagagattgaaaaaactcaaattcttacaaaactgaactgaaataaaacaagagatgcataataaaaaaatacaaatcttgatacttattacaactggaatatgcgactaagctgaaataaactgacattaaagacatgacagaaatacatgttttggtctataacaacctaagtagaaatcgcagcctgttgatgcacagatacatgtattcttatatatttgtattaatattttaatttatgtacatatcgagatgaatgaaaataagctaatatttgtcatttagaaattattccaaaactgaatcatcttacttgagttatttaattaataaaattaaatatatttcattaaacaatgcattttctctttaaaacaatccatttgaatcttactttataggaagaccttgatagataaacaatgtattggatatacgttttttttagatgctctaaagattgcattagaatgaaataaaactttttacttttaagatttttgctctttaaaataaaaagtacaaactttgataagttttatataacctaaaaagaaattgtaacatgtgagtttgagatgtcttatataatctacaaagatatttaaagagtataaacagtaaagcaataatacttaggtgatgaaaatacctcacttagtactaatacaacaaagcaagatatcgaacaaatcttaaatctttgcaaaactgaactgaagtaaaacaagaaatgcataataataagacaaatcttggaacttattatagctggaatatgccagtaagctgaaataaattgatactaaggacacaacagaaacacatgcgtctgcaacctaagaagaagttgcaacctgtcgatgcataaatatacatatgcgacgtatattgctatgtatgaaaataagcgaatacttatcatttggaatttattccaaaattggatcatcttgctgagttattcaattaataataatgaatatattttattaagaaataaaaaatttaccttttcctctttaaaacgatttatttgaatattactttatgagaagactttttataatcagaaaacttgatatataaagaatgtatttgataatgtttctttagagatgctctaaagattgtattgcacttaaataaaaccttttactcttaatagtttagtatttctcaaaattattactctttaaagtaaatagctcaaactttgagaagtcttatgtaaactaaacaaaaattgtaacctacgagtttgagatgtcttatacaacctaaaataaaatttaaagaatataaactttaagatgtctttttctatataatcaattttattttgaaaaaccaccaaagcaaaaaaaatttaggtgatggaaatatcctacctagaactaataaaatatagcaaaagattaaacaaatctcaaattcttataaaattgaactgaagtaaaacaagagatgtataataacaaattttggtacttaatacaaaaatatgtcattaagctaaagtaaagtgataataaggacacaatataaacacatgctttggcctccagcaatctaataagaaattgcaacctatcgatgcacaaatacacgtatctttatatgcttatataagtattttaatttgtgtacgtgtattcaaatgactgaaaataaactaaaacttgtcatataaaattttttcaaaattaaatgatcttgctaagttattcactaataataataaatatatttcattaaaaataaaaatttgtcctttctctataaaagagtccattagaatcttactatatatgaaggctttttataattaaaaaacttgatatataaagaatgtattggaggtgcattttttttttgagatgttctgaagattgcattggactacaataaaagtttttacttttacttcttagaattcttgctctttaaaataataagcacaaactttgagatgtcttgtagaacctaagaaaaaaattacaatttgtgatttttagatgttttatacaatctaaaaagagatttgaagagtataaattttaagatgttttatacaacctaaaacaaattgtaatctgtcgacgcacagacacgtatttttatatatttataaagatcattttaatttatgtacttatatttagataaataaaaataagctaatatttgtcatatataaattatttcataactgaattgtctgatttgttagtgttattaaattaataataatagttatattttattaaataataaaagatttaccttttctctataaaaaggggaagaaatgtggttactaaaaccatctcatattcctccgctttcttcctcagctgctcactttgtaagtattacgactttctcaaacttcctacttgttttcaattaatgttattctacctgacttcatatatatctcttttcttttgggatcattattactgctactgtaatgatcgagaaaaatctcttcttcttatcattatgacttcatatattatcatcttctatcacttcatataatgtttttgattttatgattcaatatatttataattaagttctatctttaaacgagagcaaccaattaaagaaacaagagtaaatatatataaattgtaaggtattatttgtttgatttaaatcattaaatagctagatcttctttttcttttctttttttctttctcatccaaagtttttatgaattgcaggctattagtaatatagatggtagaaagaaaattaaaattaaaacttcttcaatcatcacaaatgagagtaccaactaaactatgtgattttggtaaaaaatgcaaaacaagtacttatatatatatatatatatatatatatatatatatatatatatatattcttttataaatcagtatatacattttactctgattaccataatattatagattttactaaggtgacactaatgttatatattttggttagaatggtagtgttttcttttcttaaagatgctctagaggattcttaacaaaagaatataatatataaataatatattaaagatgccctagaaaatgcatttactgtacttaaataacctgttttgctcttaatattttattatttttatttctataataaaaaatattttaagaaatatttaagtataaaaaataaagtattttattgatgtccactgtactttttatattttatttcttattttacttttgtttccaagggcatcaatatcttcttcttttttctgtttaatttttattaataaaaaaaataattacaaatattaattaatcaaatacatagaaatttatttttataaaaaaaatccttcaaattcttttaaaatgtcattttgaccctaaatttcttttaatagttagtgttctaataaaaaaaatttacccaataatttttctaatatttcattattcttttataagacaaactcttagcctctagaattattttaaggatatatataatttgtctctctttctctttaacatagccttagtttccaataaataaataatgaaatatatttcactcttcatttctttaaacttcttacatttttttttgtagcattctttgtaagtggaatgacaaaaccgttaatgatgttcttttaaaagtgaaagatgtttatatattgcagtacagataatgatatatctactgcactacataaaacaatttaaatctccctgtttattttaagaagttatattttctttctttctcatcctaagaaagttaaattactgtaatcgacattatatgaattttaactaattccgtttctaatttataattatttcgttaaaccaatcaattccctttaaacactgcttatgcatattctgtctcaatttatatatggcatgcatcttccgtattaatttataagttcatttttattgatcaagtatttgtggttttctttatataaaaaaatgtattagtgtttttctgtattaattttataagttcatctttatgagaatgctaatgtatttggacagccaataaaattccaagaattgctgcaatcaaagatgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgccatgaaaatagatggcttaaataaaaaggacattgtaaattttgtaactgaaaggacagcaagttattgcagtccagtatctaataagagcacaactattgtcttgtgcattctaaatttatggatgaattgtatgaattaagctaattattttggtcatcagacttgatatctttttgaataaaataaataatatgttttttcaaacttataaaactatgaatgatatgaatataatgcggagactagtcaatcttttatgtaattctatgatgataaaagcttgtctcttaacttagtgaatttgtatccaagtaaaaaacagcctactaagtcatggattccttcaaatttacgctcttattataagcttaattttcatccacgatcatccctattcatgtgatgcacaagaacttaaggtatatagatttgaagtaattccttaattataatttttaagtttatcactttctttactttctaatttctttttctcaaattgtactaattaactcattgaagaatttaacaacttgttcatcagttccatagtaattctcaataaatttatggtctcacaactcaagcatccatgggttcataagttgtgataatcaggcagcttcatatatattgaaattaacataaggagtaaagtgggttgagcaatgccaggtggtacatccaggtggtacatccatatatacttcttcctccaaatcaccatgtaaaaaagtattttttacatcaaattgtcttaatgcccatcctttagttgctgcaatagaaatcagtattcaaatcgtaccaagtttcgccacaggtgcaaacgtttcctggtagtcaattccatacttctgagtaaatcctttcgctaccagtctcgcttttcgcctgtctagactcccatctgccttaagcttcactgtatagatccatttacagccaatcgtattcttcccctctggtaatttcacaaactcccatgttgcattcttctctagggctttcacctcttcctccatcgcctcttttcatttcaagtcttgcattgcctcctgcacactattaggaatcgcaatcccggataattcactagtgctaagtgcataataatctgttaaatgattatgtcgaataccattcttagttaaaaaattacttaaatgactatccataaattcctttgcattatcagtttgccaaactaaaacccatgtacgatattgatttctaataaaatttcaaaaatcacaaaaagctttacttacctcacttttgttccgaagtaaggttatccaagtcatacgagtacattcgtctataaaagttccgaaatatctagcacctgataatgaaggaatttttgcaggaccccaaacatcagaatgtataacagcaaatggttcagtacttttattaaaactaggcaaataagtagcacggtggctcttaccaagttcacacacatcacaattaaacacggaatcatccaaattaacaaacaattcaggttttaattttttcaaataactaaatgacaaatgtccaaggcgtcgatgccatagccaaattctttccattgtattacttaagctttcagtcgtgtaaacctcacttattttcttctctcctatctcagtcaagtctagataataaagtttgcctcgtttaacaccataaccaagagtctcccgagtcaggatgtcctgaaaaacacaatatgaaggccagaaagttacagtacagttaagagacgaggttatttgtccaaccgagagtaagttacaagacagtgtaagaacaatcaatacagattttaaattcagattttttgaaagagtaatggatccttctccactaacaatacatttggatccatttgttattgacacggtaaaatgagaggatggaatgaaagaataaattcgatcagaattgcataccatatgatcagatgcaccagggtctattgtccacaatgttcacacagcaagttacatttttttccagctccatttttcctgttttttccataattctggcaaacctgaaatacagtaggctccacagttgcatttgttcctcccattgtcttacattgttgatcttctcttcgaacataagcatatgtttgttctaaatcaagtttagggtctttgcgcaaaatctcacctcggacttgatcaaagtttgaatcaagcccactgaggaagatatgcatgcgtagccttgccatggcagaatgcaacgctactactccatccactgtaccttcatgtgactgagtacgatgatctatctcttgaaagatttccatcaactcagagtaatacgtaggcaatggtctgccctcttgtttgattgcgaaagacttctgattcaattcgaataacctcgtttcatcagttccatcataaaatgtttttgctgctgcctcccaaacatctttggcagtaggaagtctgatgaaatgttgcatcagtgaaggtatcatcgaatcaatcagccagctctttaccttttgattctcagttatccaggtagagtatctcgtatctgtagttgcaggcttcacttccgatccggttaaatacccagttttatttcgtgctccaatacgcatctccatgagctgtgaccatagagaataattcgattcatccagtataacactggtagggaatgatacattatcttgttgaatgataatttgattcgaagacggattcatgatttgagtatgaggtggatcttctgtactcataattctgtttagggattcagttttgatttgaaaaaaatttgttttcagtatggtgagcggctgttgtgataccatgtgagtaatgaaactctaataattgattaattttcatcctttttctctattcattcaacctgaatatatacaaaagtttgttacaaggataacttctaactacttatgtaacaagcaaccttatcaaaaatacaattagaataacatctaactactacagcttatacatacttatctctatactaacaaattaactaaatatagttaacaagttctgaatcatagtaattttagtctctataatattaacaaatgagggactaaaatacttttcttttcttaaatatgattctattttaaaaagcgggagatagaaaaatttatataatttcttactaaaaaaaagtaaggataattttatttaattctaagaaacaaagcaaaggaaaatgggaaagtttataatttgatctaatttctttgcatgatagacaggtaactgattctcaatacgattctaagatgatccaatataaattatttgtgggaaataggttcacgaaaaagccatttgttggggattcaatgtttattgatttgttttgtgtagtatgttttgcatcaattaattagatcagctagtcatttacatattcttgcaaatttgtgccatttacaaatgaattatctgaatttagtgtaatttgattaatttaattagacgggttttccttcttcagtaatcaacatacacaaagtgaaaggcaaacgagttctagcaatttaattgattaggttcaagttaattaattaatttgataatttatggttcagtccgttactgaaaatttaaaaagaagtagcatgcatctttagattctttctctattttatgcatgccattttctgattaattgtatatgttcgttcttcttgattaagtatttatatttattttttatttgattagtgagctatatatatttttaagagaatcttgatgtatttggacaactaaaaaaattccaagaattgctacaattggagatgaaactgaaaggaaacactatggtgtggatatacgcagtggcaacgtggctctgcttgggatcagccttggggtggtaattcaatgccactgtaagctacacattcccaactgtaagctttaccactgttgatgccacggagcaaatgtacagagagtgttgtagaaaataagaataagtatatatacttatcggcttagtagccaagaagtcttttggtaacctacaactcttaaacatcatatacttaatacataagttacacgcttaatgaatatatatgtaacttacatatttaataggtgactctttaatttcataacttaagcattaaaaactatacataatgattattaaattaaatttagtttttcaacactccttaaatttaatttcagatcaaaccgagtcttgctcttaatctggcaaaatcttcaaactttaatgccttcgtcaaaatatcagcaagttgatcttgcgacttgacatacttcagctctatttccttcttacggatactttctctgatgaaatgatatcttctattaatgtatttactcttaacatgtaaaataggattcttgctcaatgctatagacgatttattatcaataaaaatatcaattggttcttcgtttgaaaccaatagcacctctaacacctttcttaaccatgtggcatgacaaacacaagcagcagcaacaacatacttaacttcacaagttgaaagaattacaatagattacttctttgaactccatgtaaaagcagtctctccaatagaaaacacaaaacccattgtactctttcgatcatcaatatcacctcctcaatcactatcactatagcctactagactgaaactttttgaaggtgagtaaaacaaaccataatctaaagttccttcgacatatctcaaaattcttttaccagccttcatgtgcgaatctgttggtgcctccatgtagcgactaataagtcctactccataaagaatatcaggtctagtacaagtcaagtacctcaagtttccaacaaatctttgaaacaaaattggattcaccttctcagtattatcatatcttgacagctttactccacattcaactagtgtgctgacaagctgacattgctccatgccaaactcctttaagatctttcttacatagctcttgtgagaaataaaaatgccatcattagtttgtttcactttgattcccagataataagacatcaaccctatatctgtcatctcaaattcagcagccatctcctttttgaattattcaatcattgctgaattatttttctgtaaagatcaaatcatcaacatacaagcacaccacaagaacatcaccattctctttcttcttagaataaagagaatattcatgaggatatttcagaaaatttttctttttgaaataatcatcaatcctgttgtaccatgcccttagtacttgcttcaatccatacaaaggtaccttcagttttaaaactttgttttcttcccctctaacaacatatctgaaaggttgttttagatatccttcttcttctaagtatccattcaagaacgccgatttcacgtccatttgaaaaattcttcatctcttttgtgcagctaaagcaatgattaaacaaattatttccaatcgagcaactactacaaagacttcatcatagtcaattccatgttgttggctaaacccttttgcaacaagtcgagccttgtatttctcgacatctccgttttcattctttttaactttttataaatccatttcacaccaacaacttgcttgccttaggaaagattgctaattcccaagtgtcattcttttgaattgattttatttcgtcatctatggctactctccatttttcatttttcatcgcttcttcaaaattcattggttcagtatctgcagaaatacaataatgaacaaaatctaaaacttcatcaatgttagcatatatgtcctacagatttctcatctttgtaggtttttcagatgatcttgaacgacttgaatctccttgaatacttgctggagttcttggaagtgtcaagttctgtacatcaaaattaaaatgttgaacatcttgatctgaataatcaaaaattggaaaaaaattataattttctgattgattctcccaatttcatccatcagcttcctcaaacttgacatctcgactcaggataatctttttgcttgtcggattgtacaacttgtagactttagactttgcatcatatccaacaaaaatgaattttccacttttatcatccagcttaattctggtttcatctgcaacatgaacataagcaacataaccaaaaactctaagatgagaaagagttggctttcttccactccatgcttcctgtggtatagacttctgcaaactctttataggacatatg

SEQ ID NO: 39 - DNA, 1425 bp, synthetic construct
  misc_feature 1..1425: note = bst1 promoter
  source 1..1425: mol_type = other DNA
acgccgttttcccgcctcagcctcccgagtagctgggacaacaggcgcccgccaccgcgcctggctaatcttttgtatttttagtagagacggggtttcaccgtgttagcaaggatggtctcgatctcttgacctcgtgatccacccgcctcggcctcccaaagtgctgggattacaggtgtgagccaccgcgcccggccaatcctggctctcttaaactgaacatctccaggattttgcatctatcaggaagggatattgtgacctctgacaagttacagtcggcggtaggtatccatggagaattggttccaggaacctcctggataccaaaatccacagatgctcaaggcccttatattaaatggtgtagtatacagtatttgcacatcctcttctataattcacgtcatctccagattccttgtaatatcaagtactatgtaattgttacataagtagttgttacattggattatttagggaatgatgacaaggaaaaaaaagtccgtacatgttccgtacagatgcaagcattctttttttcctcaaatgtttctcatccgtggttagctgaatttggatgatgaatccgcaaatctggagggccgaacatgcttaacttccctgagtttatccattgctaaagcgagagaaattacaaacttagctaggaagggtgttgcggaaagtaattaatattatacttgcaaagtgcttggcacgttgttgtggttattatttcctgaaaacaaacaaacaccacttattggttggtgtgaaagagggcatctgtcaaagttgtcgtcctttggagccagaaaagtatttcttctgatgatgactgacctctcctcacctctccttcaaccttgcggcttctttggggctccctccccgtggacagcggtcaagattagcctgggccacagtgcactggtcctgtcctgggtgatgttccaagctcactcttttttaattttatttatttattaattttttttttagacagagtctcactctgtcgcccagggctggagtgcagtggtgcaatcactgctcgctgcagcctcgatcccctggggtcaagcctcctcacatctcacctcgcaggtagctgagactacagatgcgcaccaccaggaccagctaagttttgtatttttttgtagagaggaggtttctcacctgtatgttactcaggtttgtctcgaactcctatactcaagcgatccgcccgtctaggcctctcaaagtgctgggattacaggcatgagccgccgcgccccgccccacgctcagtcttgaaattgtctggaacgggaaacggcaaacagcgagatatccgagcgagagtcccgccctgcatcagtttgcggaaccgccttggtagaaggagagaaggggagtggaggaagcacggga